(19) United States
(12) Patent Application Publication
(10) Pub. No.: US 2023/0346990 A1
(43) Pub. Date: Nov. 2, 2023

(54) HIGH MOLECULAR WEIGHT HEPARIN COMPOSITIONS AND METHODS FOR DIAGNOSING, TREATING AND MONITORING EOSINOPHIL MEDIATED INFLAMMATORY DISEASES

(71) Applicant: University of Utah Research Foundation, Salt Lake City, UT (US)

(72) Inventors: Gerald J. Gleich, Salt Lake City, UT (US); Russell Morris Condie, Salt Lake City, UT (US); Debra Eckert, Salt Lake City, UT (US); Kristin M. Leiferman, Salt Lake City, UT (US); Kathryn A. Peterson, Salt Lake City, UT (US); Hedieh Saffari, Salt Lake City, UT (US)

(21) Appl. No.: 17/796,494

(22) PCT Filed: Feb. 10, 2021
(86) PCT No.: PCT/US2021/017453

§ 371 Date: Jul. 29, 2022

Related U.S. Application Data
(60) Provisional application No. 62/972,224, filed on Feb. 10, 2020.

(51) Int. Cl. A61K 51/06; A61K 31/727; A61K 47/55; A61K 9/00; A61B 6/00

(52) U.S. Cl. CPC A61K 51/06; A61K 31/727; A61K 47/55; A61K 9/0053; A61B 6/4057

(57) ABSTRACT

Disclosed herein are compositions comprising high molecular weight heparin. The compositions comprise an effective amount of high molecular weight heparin having an average molecular weight from about 20 kDa to about 40 kDa and having a purity of at least 50%, and a pharmaceutically acceptable excipient. Disclosed herein are also methods of treating eosinophil-related inflammation in a subject.

### CROSS REFERENCE TO RELATED APPLICATIONS

This application claims the benefit of the filing date of U.S. Provisional Application 62/972,224, which was filed on Feb. 10, 2020. The content of this earlier filed application is hereby incorporated by reference herein in its entirety.

### FIELD OF THE INVENTION

The present disclosure relates generally to compositions comprising high molecular weight heparin and methods of using the same. The disclosed subject matter may be applied for imaging, diagnosis, monitoring, and/or treatment of various conditions. For example, the compositions and methods disclosed herein may be used to image, diagnose, monitor, and/or treat eosinophil-related inflammation and eosinophil-related conditions such as eosinophilic esophagitis.

### BACKGROUND

Eosinophilic esophagitis (EoE) is a chronic disease of the esophagus affecting people worldwide. Symptoms include dysphagia (difficulty swallowing liquids or solids or both), food impaction (solid food sticks in the esophagus), odynophagia (painful swallowing), heartburn, chest pain, asthma, diarrhea, and vomiting. While present in adults, the disease can also manifest in children. The symptoms of EoE resemble an atopic allergenic inflammatory condition of the esophagus, affecting up to 10% of adults presenting for upper endoscopy.

Although the source or sources of this disease have not been conclusively identified, investigators have identified several contributing factors. Genetic predisposition may be at work in this disease, at least in part, due to the increased incidence in first degree relatives of EoE patients relative to the general population. Environmental causes may also be important, however, EoE is not simply a seasonal allergy of the esophagus. Despite current treatment with swallowed aerosolized steroids, the response rate is little better than 50%.

Food hypersensitivities also play an important role in both adult and pediatric EoE. However, data on an elimination diet in adults found less robust responses than those observed in children. In EoE, the inflammation occurs in various parts of the esophagus; there is approximately equal incidence in the proximal, distal, or both portions of the esophagus being affected within cohorts, but such infiltrate varies in each individual with many demonstrating a less intense infiltrate proximally. EoE also affects the luminal structure of the esophagus. The areas of inflammation are not evenly distributed throughout an affected esophagus, as the disease often presents in patches or select segments of the 25-30 cm long adult esophagus. Pronounced rings or furrows can develop into strictures that close off the esophagus, resulting in odynophagia, dysphagia, food impaction, and emergency hospital visits. Accordingly, expeditious treatment of EoE is important to alleviate symptoms before they exacerbate and restrict the esophageal lumen.

Although EoE can be diagnosed by esophagogastroduodenoscopy (EGD), some cases may never present as a “ringed-esophagus” during EGD and may be difficult to diagnose by this method. A conclusive means currently available to clinicians to positively identify EoE is to detect the presence of eosinophils in biopsy specimens. Tissue samples may be collected during EGD and then examined with traditional histological analysis to confirm or reject a case of EoE. However, the patchy nature of the disease complicates collection of tissue samples for biopsy. When clinical suspicion for EoE is high, consensus practice requires sampling at 4 to 5 sites throughout the esophagus and this still might result in under diagnosis of EoE if mucosal eosinophilia is particularly patchy. Accordingly, there is a need for more accessible methods of diagnosis and monitoring.

Despite the rapidly growing incidence of EoE, state-of-the-art diagnostic techniques remain inadequate to fully characterize this disease. Further, available treatments are not able to adequately target and localize to eosinophil-related inflammation. As such, there exists a need to develop a non-invasive, precise, and comprehensive technique to image, diagnose, monitor, and treat eosinophil-related inflammation and conditions such as EoE.

### SUMMARY

This summary is provided to comply with 37 C.F.R. § 1.73. It is submitted with the understanding that it will not be used to interpret or limit the scope or meaning of the present disclosure.

Disclosed herein are compositions comprising an effective amount of high molecular weight heparin having an average molecular weight from about 20 kDa to about 40 kDa, wherein at least 50% of heparin chains in the heparin have a molecular weight of at least 20 kDa; and a pharmaceutically acceptable excipient.

Disclosed herein are methods of treating a tissue exhibiting eosinophil-related inflammation in a subject, the method comprising administering to the subject a composition comprising a therapeutically effective dose of heparin having an average molecular weight from about 20 kDa to about 40 kDa, wherein at least 50% of heparin chains in the heparin have a molecular weight of at least 20 kDa, and a pharmaceutically acceptable excipient, wherein the heparin binds to one or more eosinophil granule proteins in the tissue to reduce the eosinophil-related inflammation.

Disclosed herein are methods of reducing eosinophil-related inflammation in a tissue, the methods comprising: administering to a subject a composition comprising a therapeutically effective dose of heparin having an average molecular weight from about 20 kDa to about 40 kDa, wherein at least 50% of heparin chains in the heparin have a molecular weight of at least 20 kDa, and a pharmaceutically acceptable excipient, wherein the heparin binds to one or more eosinophil granule proteins in the tissue to reduce the eosinophil-related inflammation.

Disclosed herein are methods of producing a medical image of an organ in a subject, the methods comprising: detecting an eosinophil granule protein in the mucosal tissue of the organ in a subject, comprising administering to a subject radiolabeled heparin under conditions wherein the radiolabeled heparin binds to an eosinophil granule protein to form a radiolabeled heparin/eosinophil granule protein complex, and detecting the radiolabeled heparin/eosinophil granule protein complex in the mucosal tissue of the organ, whereby detecting the radiolabeled heparin/eosinophil granule protein complex in the mucosal tissue of the organ produces a medical image of the organ in the subject. In some aspects, the radiolabeled heparin comprises heparin having an average molecular weight from about 20 kDa to about 40 kDa, wherein at least 50% of heparin chains in the heparin have a molecular weight of at least 20 kDa.

Disclosed herein are methods of diagnosing eosinophilic esophagitis in a subject, the methods comprising: detecting an eosinophil granule protein in the mucosal tissue of the esophagus in a subject, comprising administering to a subject radiolabeled heparin under conditions wherein the radiolabeled heparin binds to an eosinophil granule protein to form a radiolabeled heparin/eosinophil granule protein complex, and detecting the radiolabeled heparin/eosinophil granule protein complex in the mucosal tissue of the esophagus, whereby detecting the radiolabeled heparin/eosinophil granule protein complex in the mucosal tissue of the esophagus diagnoses eosinophilic esophagitis in the subject. In some aspects, the radiolabeled heparin comprises heparin having an average molecular weight from about 20 kDa to about 40 kDa, wherein at least 50% of heparin chains in the heparin have a molecular weight of at least 20 kDa.

Disclosed herein are methods of detecting eosinophil degranulation in a subject, the methods comprising: detecting an eosinophil granule protein in a subject, comprising administering to a subject radiolabeled heparin under conditions wherein the radiolabeled heparin binds to an eosinophil granule protein to form a radiolabeled heparin/eosinophil granule protein complex, and detecting the radiolabeled heparin/eosinophil granule protein complex, whereby detecting the radiolabeled heparin/eosinophil granule protein complex detects eosinophil degranulation in the subject. In some aspects, the radiolabeled heparin comprises heparin having an average molecular weight from about 20 kDa to about 40 kDa, wherein at least 50% of heparin chains in the heparin have a molecular weight of at least 20 kDa.

Disclosed herein are methods of delivering a therapeutic agent to a diseased organ, the methods comprising, administering a therapeutically effective amount of a composition comprising heparin conjugated to a therapeutic agent to a subject. In some aspects, the heparin has an average molecular weight from about 20 kDa to about 40 kDa, wherein at least 50% of heparin chains in the heparin have a molecular weight of at least 20 kDa.

Disclosed herein are methods of treating eosinophilic-related inflammation in a subject, the methods comprising, administering a therapeutically effective amount of a composition comprising an effective amount of heparin having an average molecular weight from about 20 kDa to about 40 kDa, wherein at least 50% of heparin chains in the heparin have a molecular weight of at least 20 kDa; and a pharmaceutically acceptable excipient to the subject.

Disclosed herein are methods of treating eosinophilic-related inflammation in a subject, the methods comprising, administering a therapeutically effective amount of a composition comprising heparin conjugated to a therapeutic agent to a subject. In some aspects, the heparin has an average molecular weight from about 20 kDa to about 40 kDa, wherein at least 50% of heparin chains in the heparin have a molecular weight of at least 20 kDa.

### BRIEF DESCRIPTION OF THE DRAWINGS

The accompanying figures, which are incorporated in and constitute a part of this specification, illustrate several aspects and together with the description serve to explain the principles of the invention.

FIG. 1 shows coronal and sagittal images of single-photon emission computed tomography/computed tomograph (SPECT/CT) scans obtained 1 hour after oral administration of 99mTC-heparin for patient 1 with gastroesophageal reflux disease (GERD) and patients 2 to 5 with EoE. 99mTc-heparin localization is red in the images and is apparent, and prominent in Patient 3, in the esophagus on the sagittal images (red below the diaphragm is in the stomach and/or intestines). Esophageal localization of 99mTc-heparin was not evident in Patient 1 (without EOE) or in Patient 4 (with treated EoE and absent inflammation). Esophageal binding is less intense and less continuously present in Patient 5, but, nonetheless, apparent in FIG. 2.

FIG. 2 shows images of single-photon emission computed tomography (SPECT) scans on five patients (coronal views Patient 1, 3, 4, 5, and partially rotated view for Patient 2) one hour after oral administration of 99mTc-heparin. Images were obtained after patients had swallowed 99mTc-heparin over a 15-minute time period and then swallowed 100 ml of water (as a wash to remove weakly bound 99mTc-heparin). The images include anatomical fiducial markers, including suprasternal notch (evident on images from Patients 1, 3 and 4 and more faintly on Patient 2), right shoulder (on image from Patient 5) and breast/nipples (evident on the images from the other Patients, except for Patient 2 where left breast/nipple marker is obscured). Esophageal localization of Tc99m-heparin is clearly evident in images of Patients 2, 3, and 5.

FIGS. 3A-B show eosinophil granule major basic protein-1 (eMBP-1) immunostaining of proximal (FIG. 3A), and distal (FIG. 3B) esophageal biopsy specimens from each of the five patients (200×microscopy view).

FIG. 4 shows the analyses of heparin binding to immobilized MBP by surface plasmon resonance. RU (ordinate) refers to response units, the measure of binding of heparins to MBP. Note that the most intense binding is by heparins eluting from the BioGel P60 column in peak 1 close to the first detectable fractions. In contrast, heparin in peak 2 bound poorly to MBP. Col6 early peak 1 eluted at volume 34 mL and Col6 late peak 1 eluted at 93 mL. Immobilized: EMBP-1 (2100-2800 RU); and Analytes: 0.216 ng/mL heparin fractions.

FIGS. 5A-D show the analyses of heparin binding to immobilized MBP by surface plasmon resonance. RU (ordinate) refers to response units. FIG. 5A shows varying concentrations of unfractionated heparin using pharmaceutical grade heparin (commonly employed for patient anticoagulation treatment). FIGS. 5B-D show binding of differing concentrations of fractions from the BioGel P60 column (FIG. 6). Immobilized: EMBP-1 (2100-2800 RU) (FIG. 5A, unfractionated heparin; FIG. 5B, Col6, late peak 1; FIG. 5C, Col6, early peak 1; and FIG. 5D, enoxaparin).

FIG. 6 is a chromatogram of heparin fractionated on BioGel P60 (95 cm×1.2 cm). The heparin contained a preservative that eluted starting at volume approximately 100 ml; column eluents after approximately volume 100 ml did not contain heparin. Only eluent up to 100 ml contained heparin.

FIG. 7 shows the calibration of gel permeation column by USP molecular weight standards with retention times measured by refractive indices of the standards. The table lists the relationships between the known molecular weights of the standards and the calculated molecular weights.

FIG. 8 shows the analysis of heparin fraction 12 #2 from the BioGel P60 column shown in FIG. 6. This fraction is referred to as Col6 early peak 1. The analysis is by gel permeation chromatography as described in FIG. 7. This heparin bound avidly to eMBP1 by surface plasmon resonance as shown in FIG. 4.

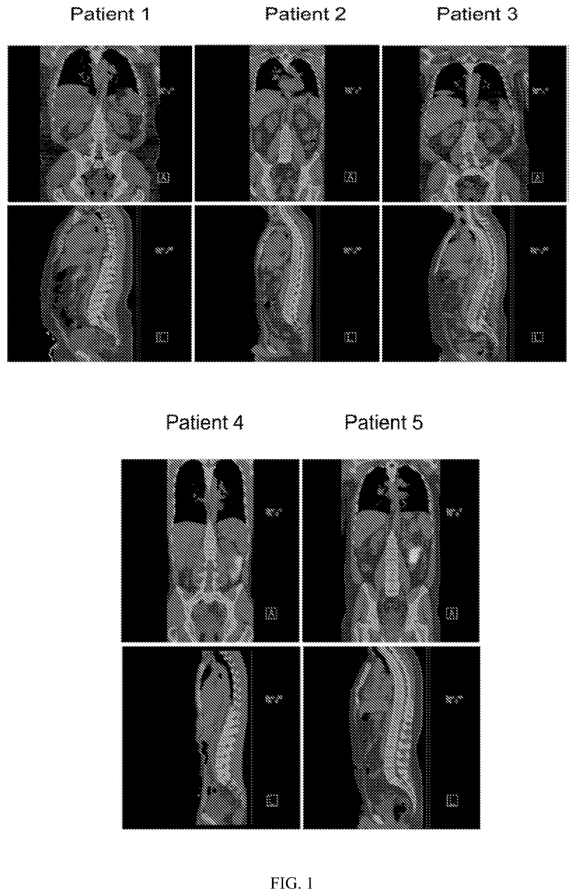

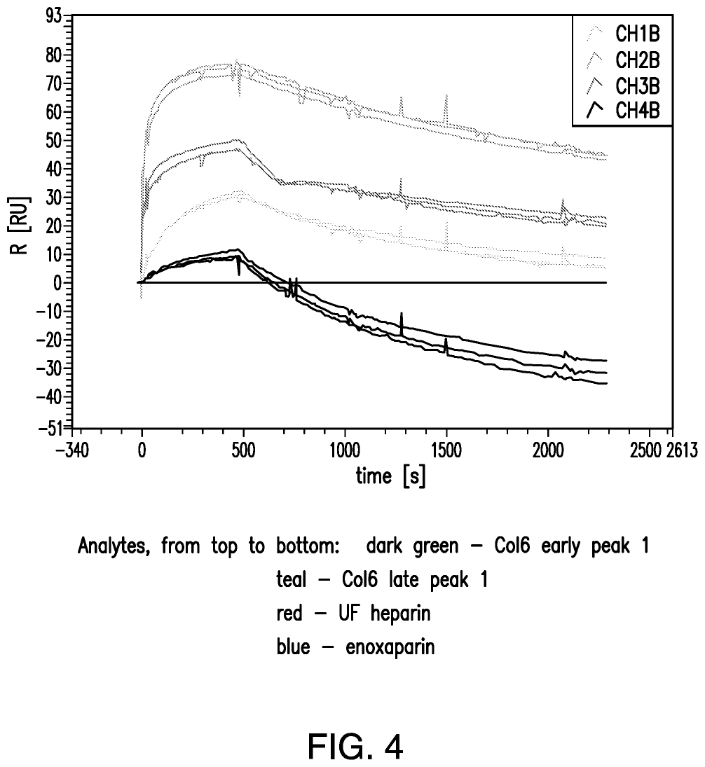

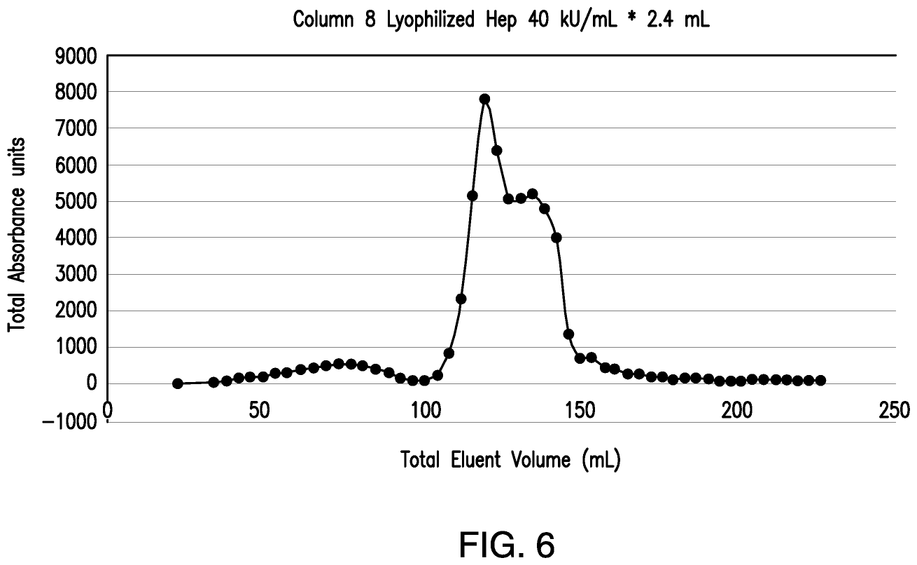

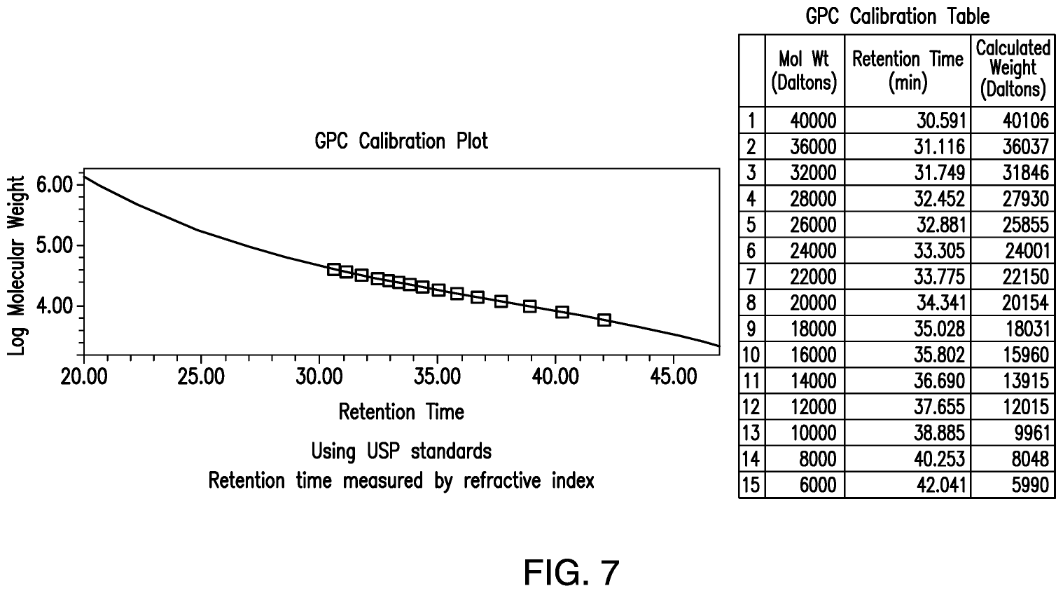

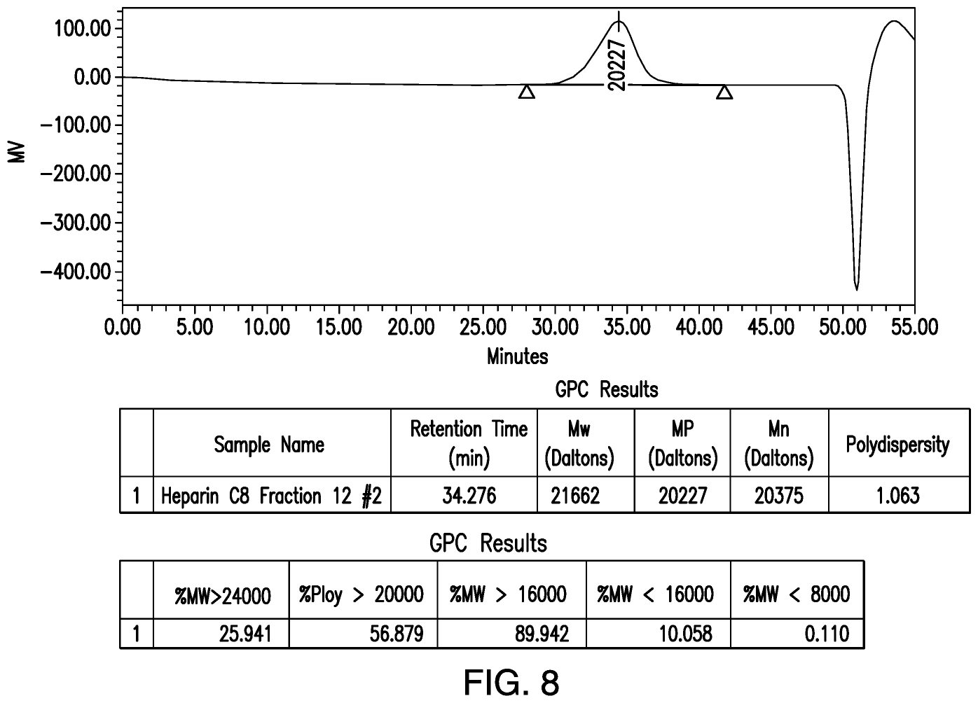

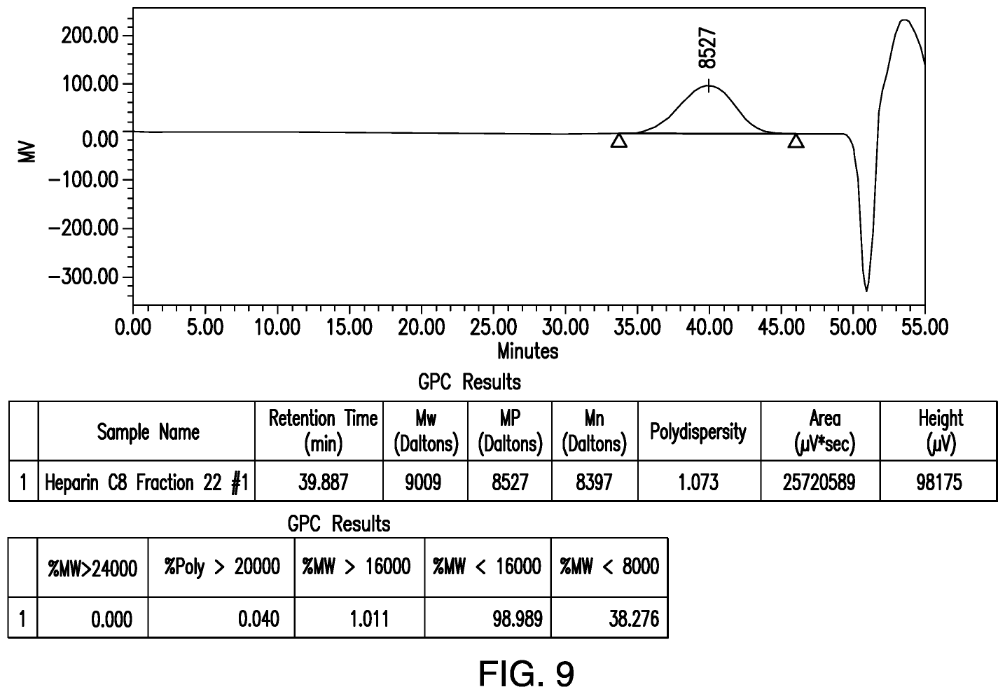

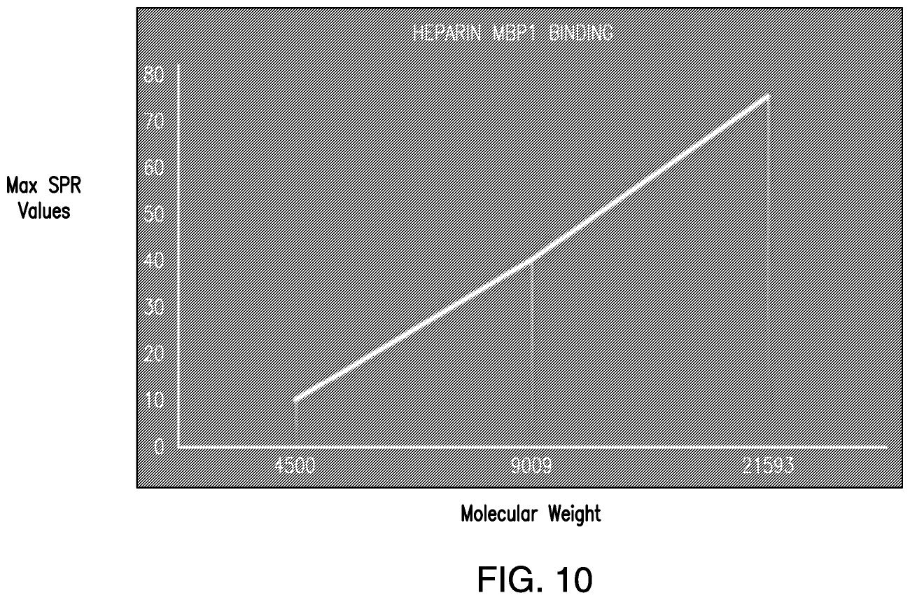

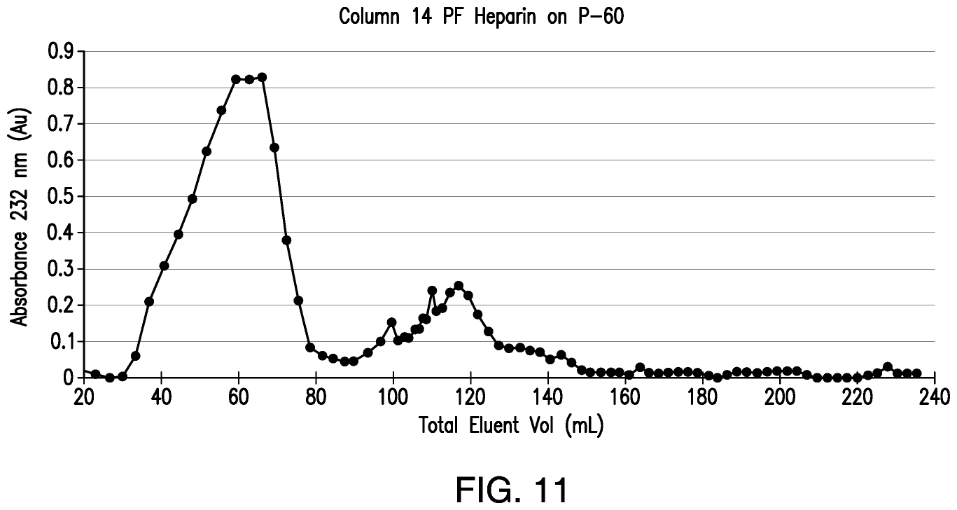

FIG. 1g. 9 shows the analysis of heparin fraction 22 #1 from the BioGel P60 column shown in FIG. 6. This fraction is referred to as Col6 late peak 1. The analysis is by gel permeation chromatography as described in FIG. 7. See FIG. 4 for its binding to eMBP1 by surface plasmon resonance.

FIG. 10 shows the relationship between heparins of varying molecular weights and maximum bindings to immobilized eMBP1 by surface plasmon resonance

FIG. 11 shows a chromatogram in which high molecular weight heparins were contained in the fractions eluting between about 33 mL to about 50 mL.

### DETAILED DESCRIPTION

The present invention may be understood more readily by reference to the following detailed description of various aspects of the invention and the Examples included therein and to the Figures and their previous and following description.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the disclosed methods and compositions, the particularly useful methods, devices, and materials are as described.

This disclosure is not limited to the particular systems, devices and methods described, as these may vary. The terminology used in the description is for the purpose of describing the particular versions or embodiments only, and is not intended to limit the scope. Such aspects of the disclosure may be embodied in many different forms; rather, these embodiments are provided so that this disclosure will be thorough and complete, and will fully convey its scope to those skilled in the art.

As used in the specification and the appended claims, the singular forms “a,” “an,” and “the” include plural referents unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

Ranges may be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant, both in relation to the other endpoint and independently of the other endpoint.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof Δny listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

All percentages, parts and ratios are based upon the total weight of the topical compositions and all measurements made are at about 25° C., unless otherwise specified.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

As used herein, the word “comprise” and variations of the word, such as “comprising” and “comprises,” means “including, but not limited to” and is not intended to exclude, for example, other additives, components, integers or steps.

As used herein, by “subject” is meant an individual. A subject can be a mammal such as a primate, for example, a human. The term “subject” includes domesticated animals such as cats, dogs, etc., livestock (e.g., cattle, horses, pigs, sheep, goats, etc.), and laboratory animals (e.g., mice, rabbits, rats, gerbils, guinea pigs, possums, etc.). As used herein, the terms “subject” and “patient” are interchangeable.

As used herein, the term “therapeutic” means an agent utilized to treat, combat, ameliorate, or improve an unwanted condition or disease of a patient. In part, embodiments of the present invention are directed to the treatment of eosinophilic-related inflammation.

The term “effective amount” is employed herein to refer to an amount of a compound that, when administered to a subject, is appropriate for carrying out a purpose of the compound including imaging of a tissue of the subject, diagnosing a disorder in the subject, and/or monitoring of a symptom or disorder of the subject. The actual amount which comprises the “effective amount” will vary depending on a number of conditions including, but not limited to, the severity of the disorder, the size and health of the patient, the imaging modality, the manner of diagnosis, the manner of monitoring, and the route of administration. A skilled medical practitioner can readily determine the appropriate amount using methods known in the medical arts.

The term “therapeutically effective amount” is employed herein to refer to an amount of a compound that, when administered to a subject, is capable of reducing a symptom of a disorder in a subject or enhance the texture, appearance, color, sensation, or hydration of the intended tissue treatment area. The actual amount which comprises the “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g., animals), and more particularly, in humans.

Where this disclosure makes reference to the term “doctor” and additional terms for various medical professionals by specific job title or role, nothing in this disclosure is intended to be limited to a specific job title or function. Doctors or medical professionals can include any doctor, nurse, medical professional, or technician. Any of these terms or job titles can be used interchangeably with the user of the systems disclosed herein unless otherwise explicitly demarcated. For example, a reference to a physician could also apply, in some embodiments to a technician, nurse, or other health care provider.

The term “tissue” refers to any aggregation of similarly specialized cells which are united in the performance of a particular function.

The term “disorder” is used in this disclosure to mean, and is used interchangeably with, the terms disease, condition, or illness, unless otherwise indicated.

The terms “administer,” “administering” or “administration” as used herein refer to administering to a subject a compound (also referred to as an agent of interest), a pharmaceutically acceptable salt of the compound (agent of interest), or a composition directly by the subject or by a health care provider.

The term “treat,” “treated,” or “treating” as used herein refers to both therapeutic treatment, wherein the object is to reduce the frequency of, or delay the onset of, symptoms of a medical condition, or to otherwise obtain beneficial or desired clinical results. For the purposes of this invention, beneficial or desired clinical results include, but are not limited to, reversal, reduction, or alleviation of one or more mores symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “inhibiting” includes the administration of a composition of the present invention to prevent the onset of the symptoms, alleviating the symptoms, reducing the symptoms, delaying or decreasing the progression of the disease and/or its symptoms, or eliminating the disease, condition or disorder.

In some aspects, the compositions and methods disclosed herein can be utilized with or on a subject in need of such examination, diagnosis, monitoring, and/or treatment, which can also be referred to as “in need thereof” As used herein, the phrase “in need thereof” means that the subject has been identified as having a need for the particular method or treatment or has been identified with a condition and that the method (e.g., imaging of a tissue, diagnosis of a condition, monitoring of a condition) or treatment has been utilized with or on the subject for that particular purpose.

For example, in some aspects, the invention is directed to a pharmaceutical composition comprising high molecular weight heparin or a salt thereof having a purity of at least 50%, and a pharmaceutically acceptable carrier or diluent, or an effective amount of a pharmaceutical composition as defined herein.

The compositions disclosed herein can be administered in the conventional manner by any route where they are active. Administration can be systemic, topical, by inhalation, or oral. For example, administration can be, but is not limited to, parenteral, intraperitoneal, transdermal, oral, buccal, or ocular routes, or intravaginally, by inhalation, by depot injections, or by implants. In some aspects, the parenteral route of administration can be subcutaneous, intravenous, intradermal and intramuscular. Thus, modes of administration for the compositions of the present invention (either alone or in combination with other pharmaceuticals) can be, but are not limited to, sublingual, injectable (including short-acting, depot, implant and pellet forms injected subcutaneously or intramuscularly), topical (including ointments or creams, e.g., for application to the skin), inhalation (including nasal sprays) and/or by use of vaginal creams, suppositories, pessaries, vaginal rings, rectal suppositories, intrauterine devices, and transdermal forms such as patches and creams.

Specific modes of administration will depend on the indication or purpose. The selection of the specific route of administration and the dose regimen is to be adjusted or titrated by the clinician according to methods known to the clinician in order to obtain the optimal clinical response. The amount of compound to be administered is that amount which is effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). In some aspects, the dose or dosing regimen used in the methods disclosed herein can be the dose or dosing regiman described in Ashoor T M, et al., Nebulized heparin and salbutamol versus Salbutamol alone in acute exacerbation of chronic obstructive pulmonary disease requiring mechanical ventilation: a double blind randomised controlled trial, Korean J Anesthesiol. 2020 Feb. 28 or Hiremath M, et al., Heparin in the long-term management of ligneous conjunctivitis: a case report and review of literature, Blood Coagul Fibrinolysis. 2011 October; 22(7):606-9.

For oral administration, the compositions can be formulated readily by combining high purity high molecular weight heparin with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Pharmaceutical compositions which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All compositions for oral administration should be in dosages suitable for such administration. In some aspects, the compositions can dissolve in the small intestine.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum. Pharmaceutical compositions of the compounds may also comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

As used herein, a “mucosal tissue” is a tissue lining various cavities within the body. Examples of a mucosal tissue include, but are not limited to, mucosal tissue lining the nose, sinuses, bronchi, lungs, conjunctiva, oral cavity, tongue, esophagus, stomach, pylorus, duodenum, jejunum, ileum, ascending colon, caecum, appendix, transverse colon, descending colon, rectum, anus, urethra, and urinary bladder. A mucosal tissue comprises an epithelial surface, glandular epithelium which secretes mucus, basement membrane, and submucosa with connective tissue. Thus, a radiolabeled heparin/eosinophil granule protein complex can be detected on the epithelial surface, in the glandular epithelial tissue, on or in the basement membrane, and in the submucosal connective tissue of a mucosal tissue in a subject. In some aspects, a mucosal tissue is within or from the esophagus of a subject.

As used herein, an “eosinophil granule protein” is a protein that comprises the granules in eosinophils. When an eosinophil is activated, granule proteins are released from the cell into the surrounding tissue. The released granule proteins can cause pathologic inflammatory responses in the surrounding tissue, for example esophageal mucosal tissue. Examples of eosinophil granule proteins include, but are not limited to, major basic protein (MBP), major basic protein 1 (MBP-1), major basic protein 2 (MBP-2), eosinophil derived neurotoxin (EDN), eosinophil cationic protein (ECP), and eosinophil peroxidase (EPO). Other examples of eosinophil granule proteins are provided in Kita et al., Biology of Eosinophils, Chapter 19 of Immunology, which is hereby incorporated by reference for its teaching of examples of eosinophil granule proteins. In some aspects, an eosinophil granule protein can be MBP-1.

As used herein, “high molecular weight heparin” refers to heparin and/or heparin salts (e.g., heparin sulfate) having a molecular weight of above about 20 kDa or more. A heparin polymer typically consists of a mixture of polydisperse linear polymers, i.e., having molecular chains of varying lengths, such that the molecular weight of the heparin chains varies and cannot be fully described by a single number. Accordingly, high molecular weight heparin is more particularly described as having an average molecular weight of above about 20 kDa. Average molecular weight may be calculated as a number average (i.e., total weight of the sample divided by the number of molecules in the sample). As used herein, “low molecular weight heparin” refers to heparin and/or heparin salts (e.g., heparin sulfate) having a molecular weight of less than about 8 kDa. For example, Enoxaparin is a product in a low molecular weight heparin family and has a molecular weight of about 4.5 kDa. Heparin polymer typically consists of a mixture of polydisperse linear polymers, i.e., having molecular chains of varying lengths, such that the molecular weight of heparin chains varies and cannot be fully described by a single number. Accordingly, low molecular weight heparin is more particularly described as having an average molecular weight of less than about 8 kDa. Average molecular weight may be calculated as a number average (i.e., total weight of the sample divided by the number of molecules in the sample).

As used herein, “unfractionated heparin” or “heparin” refers to a heparin polymer with molecular chains of varying lengths, and molecular weights ranging from 3 to 30 kDa. “Unfractionated heparin” or “heparin” is polydisperse, not having been fractionated to sequester the fraction of molecules with a particular limited range of molecular weight (as is the case with high molecular weight heparin and low molecular weight heparin).

As used herein, a “radiolabel” is an isotopic composition that can be attached to a substance, for example heparin, to track the substance as it passes through a system or tissue. A non-limiting example of a radiolabeled substance is radiolabeled heparin including, but not limited to radiolabeled high molecular weight heparin, radiolabeled low molecular weight heparin as well as radiolabeled unfractionated heparin. As provided herein, the methods described herein can be used with any of the radiolabeled heparins disclosed herein, including but not limited to radiolabeled high molecular weight heparin, radiolabeled low molecular weight heparin as well as radiolabeled unfractionated heparin. In some aspects, a radiolabeled heparin can be 99mTc-heparin. Examples of other radiolabels include, but are not limited to, ¹¹¹In, ¹⁴C, ³H, ¹³N, ¹⁸F, ⁵¹Cr, ¹²⁵I, ¹³³Xe, ^{81m}Kr, and ¹³¹I. Other radiolabels that can be attached to a substance, for example heparin, can be found in Table 1. A radiolabel, for example, 99mTc, can be attached to a substance, for example heparin, using commercially available reagents well known to persons of ordinary skill in the art. In some aspects, 99mTc-heparin can be prepared as shown in Example 1 below.

TABLE 1
Commonly utilized radiolabels

| Nuclide | Physical half-life | |
|---|---|---|
| ³H | 12.3 | years |
| ¹¹C | 20.4 | minutes |
| ¹³N | 10 | minutes |
| ¹⁴C | 5730 | years |
| ¹⁵O | 2 | minutes |
| ¹⁸F | 110 | minutes |
| ³²P | 14.3 | days |
| ⁵¹Cr | 27.7 | days |
| ⁵²Fe | 8.3 | hours |
| ⁵⁷Co | 271 | days |
| ⁵⁸Co | 71 | days |
| ⁵⁹Fe | 45 | days |
| ⁶⁰Co | 5.2 | years |
| ⁶²Zn | 9.3 | hours |
| ⁶²Cu | 9.7 | minutes |
| ⁶⁴Cu | 12.7 | hours |
| ⁶⁷Cu | 2.6 | days |
| ⁶⁷Ga | 78.2 | hours |
| ⁶⁸Ga | 68 | minutes |
| ⁷⁶Br | 16 | hours |
| ^{81m}Kr | — | |
| ⁸²Rb | 75 | seconds |
| ⁸²Sr | 25.5 | days |
| ⁸⁶Y | 14.74 | hours |
| ⁸⁹Zr | 3.27 | days |
| ⁸⁹Sr | 50.6 | days |
| ⁹⁰Sr | 28.5 | years |
| ⁹⁰Y | 2.7 | days |
| ⁹⁹Mo | 66 | hours |
| ^{99m}Tc | 6.0 | hours |
| ¹¹¹In | 2.8 | days |
| ¹¹³In | 100 | minutes |
| ¹²³I | 13.2 | hours |
| ¹²⁴I | 4.2 | days |
| ¹²⁵I | 60 | days |
| ¹³¹I | 8.0 | days |
| ¹³³Xe | 5.3 | days |
| ¹³⁷Cs | 30 | years |
| ¹⁵³Sm | 1.9 | days |
| ¹⁸⁶Re | 3.8 | days |
| ²⁰¹Tl | 73 | hours |

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

As disclosed herein, EoE may present in a patient generally as inflammation in the esophagus or as more pronounced rings or furrows that constrict or even block the esophagus. EoE can cause dysphagia, food impaction, odynophagia, and other symptoms that are painful and dangerous when left undiagnosed and/or untreated. Further, eosinophil-related inflammation can occur in other organs and tissues to cause additional painful symptoms and/or dangerous conditions.

An important element for diagnosing EoE in a biopsy specimen is the presence of eosinophils. Normal esophageal tissue does not contain eosinophils. These white blood cells were named for their affinity for the red dye eosin. Normally, eosinophils reside in the blood stream, stomach, small and large intestine, and lymphatic system but infiltrate pathologically into the esophagus in EoE. Some clinical evidence suggests that inflammation increases with eosinophil concentration. A distinctive characteristic of eosinophils is their granules which comprise markedly cationic proteins. The granule is composed of an electron-dense central core and an electron-radiolucent matrix. The core consists primarily of major basic protein 1 (MBP-1); the matrix consists of eosinophil peroxidase (EPO) and eosinophil derived neurotoxin (EDN) and the eosinophil cationic protein (ECP), inter alia. MBP-1 is a highly basic (isoelectric point greater than 11) 13.8 kDa protein with 5 unpaired cysteines that accounts for about 52% (humans) to 55% (guinea pig) of the granule's protein (see, for example, Abu-Ghazaleh R I, et al., Eosinophil granule proteins in peripheral blood granulocytes; J Leukoc Biol 52:611-618, 1992). It is a member of the C-type lectin family (lectins bind sugars) and has the highest concentration in the eosinophil granule on a per molecule basis. EPO has the highest concentration in the granule on a per mass basis, while EDN and ECP are members of the RNAse 2 family. Upon degranulation, an eosinophil releases each of these proteins into the surrounding tissues. Of these, only MBP-1 stimulates histamine release. MBP-1 also exfoliates bronchial epithelial cells and causes bronchial hyper-reactivity, whereas both MBP-1 and EPO provoke transient bronchial constriction. These proteins are found in abundance in biopsies in EoE. Recent findings further suggest that the enhanced sensitivity of bronchopulmonary C-fibers induced by the eosinophil granule cationic proteins may be a contributing factor in the pathogenesis of bronchial hyperresponsiveness and chronic cough associated with eosinophilic infiltration of the airways.

Initial studies of this molecule showed that it precipitated with heparin indicating an interaction presumably based on charge. Later studies showed that MBP-1 was toxic to mammalian cells, bacteria and certain forms of parasites, and it was deposited at sites of inflammation in numerous eosinophil-related diseases in association with organ dysfunction. Heparin neutralized the cytotoxic effects of MBP-1 in a dose related manner. Still later, investigations showed that heparin interacted more intensely with the MBP-1 than did two other markedly cationic proteins, namely the eosinophil peroxidase and the eosinophil cationic protein. The affinity of heparin for MBP-1 can be due to its ability to localize to a specific site on MBP-1. Accordingly, binding to MBP-1 will neutralize the toxic effects and alleviate the symptoms associated therewith. Ideally, MBP-1 may be targeted with a composition that localizes to tissues expressing eosinophil-related inflammation and has a therapeutic effect on the tissue by binding with MBP-1.

Disclosed herein are methods and compositions that can be used to visualize active EoE in the entire esophagus and monitor disease activity. In some aspects, the methods and compositions disclosed herein may lead to a decrease in the number of EGD procedures and biopsies that patients with EoE currently require. Furthermore, the results described herein evidence that eosinophil-related inflammation in other organs may be detected by binding Tc99m-heparin; thus, in some aspects, a radiolabeled contrast agent, such as Tc99m-heparin, can be used as a diagnostic agent for eosinophil-related diseases throughout the body.

Also, disclosed herein are compositions comprising high-molecular weight heparin and methods of using high-molecular weight heparin for the localization and treatment of eosinophil-related inflammation (e.g., in the esophagus). Unfractionated heparin and low molecular weight heparin can also be used for the localization of eosinophilic-related inflammation. In some aspects, the disclosed compositions comprising unfractionated heparin can be used for the localization and/or treatment of eosinophil-related inflammation (e.g., in the esophagus). Advantages of the compositions and methods disclosed herein include but are not limited to conjugating, for example, high-molecular weight heparin with one or more glucocorticoids for direct targeting of eosinophilic-related inflammation, and treating gastrointestinal eosinophil-related inflammation and/or one or more eosinophil-associated diseases.

The compositions and methods disclosed herein have at least two advantages. First, for the localization of eosinophil-related inflammation, utilization of high molecular weight heparin will bind more avidly than low molecular weight heparin to sites of eosinophilic inflammation. In turn, the quantity of heparin (e.g., high molecular weight heparin) used for localization of eosinophilic inflammation can be reduced with the expectation that a greater percentage of radioactivity will localize to the one or more sites of inflammation. Further, using the compositions and methods disclosed herein can reduce the quantity of heparin, and, thus, the quantity of radioactivity used for localization of eosinophilic inflammation, thereby limiting a patient's exposure to radioactivity. Further, the compositions and methods described herein can be used to identify eosinophilic inflammation in and throughout the body (e.g., any organ of the human body afflicted with eosinophilic inflammation).

Second, compositions comprising high molecular weight heparin may also be more effective for neutralizing the toxic effects of eMBP-1 compared to low molecular weight heparin. In some aspects, the high molecular weight heparin will have the capacity to function as a medication by application to or delivery to one or more sites of eosinophilic inflammation. For example, in some aspects, compositions comprising high molecular weight heparin may be used to treat eosinophil-related gastrointestinal tract diseases, for instance, eosinophilic esophagitis, by oral administration of high molecular weight heparin to neutralize eosinophilic inflammation. Furthermore, because the high molecular weight heparin can be used to target eosinophil-related inflammation and because glucocorticoids such as fluticasone or budesonide can be conjugated to the high molecular weight heparin, in some aspects, high molecular weight heparin can be administered before, after or simultaneously with one or more glucocorticoids to treat eosinophil-related inflammation.

High molecular weight heparin can be effective for localizing to sites of eosinophil-related inflammation and for neutralizing the toxic effects of eMBP-1. In some aspects, the high molecular weight heparin can function as a medication by application (e.g., a therapeutic agent) to or delivery to one or more sites of eosinophilic inflammation. In some aspects, because the high molecular weight heparin can be used to target eosinophil-related inflammation, tracers (e.g., radiolabeled contrast agents) and/or therapeutic agents can be conjugated to the high molecular weight heparin to provide a targeted delivery to the eosinophil-related inflammation.

Historically, high molecular weight heparin has been avoided in favor of low molecular weight heparin. Generally, the heparin has an inherent heterogeneity in terms of lengths of polymer chains and thus molecular weight. It is typically believed that administration of significant quantities of high molecular weight heparin chains by the common routes of administration (i.e., intravenously or subcutaneously) increases the incidence of heparin-induced thrombocytopenia (HIT), a complication that results from exposure to heparin and can have limb- and life-threatening thrombotic complications. In HIT, the immune system forms antibodies against heparin when it is bound to platelet factor 4 (PF4). The antibodies then form a complex with the heparin/PF4 and bind and activate platelets, resulting in the formation of blood clots and a drop in platelet count. HIT can lead to venous thromboembolism and in some cases arterial thrombosis (known as HITT). Due to the risk of HIT suspected to be associated with high molecular weight heparin chains, low molecular weight heparin has been favored in the medical field for clinical applications. However, the risk of HIT may be greatly diminished where heparin is administered orally, by inhalation or topically as opposed to intravenously and/or subcutaneously.

Further, the lack of uniformity in chain length creates major difficulties in isolating a particular molecular weight of heparin. Although some techniques successfully produce compositions having a targeted average molecular weight, the molecular weight nonetheless varies greatly and thus the compositions do not have a high purity (i.e., a high fraction of the composition having a molecular weight in the targeted range). Given the focus on low molecular weight heparin, methods of purifying low molecular weight heparin have been explored but there has not been similar progress in developing methods of purifying high molecular weight heparin.

Eosinophil granule major basic protein-1. The eosinophil granule major basic protein-1 (eMBP1−) is a markedly cationic molecule with a molecular weight of approximately 14 kDa and is localized within the eosinophil granule to the core of the granule. Initial studies of this molecule showed that it precipitated with heparin indicating an interaction presumably based on charge. Later studies showed that eMBP-1 was toxic to mammalian cells, bacteria and certain forms of parasites, and it was deposited at sites of inflammation in numerous eosinophil-related diseases in association with organ dysfunction. Heparin neutralized the cytotoxic effects of eMBP-1 in a dose related manner. Still later, investigations showed that heparin interacted more intensely with the eMBP-1 than did two other markedly cationic proteins, namely the eosinophil peroxidase and the eosinophil cationic protein. The affinity of heparin for eMBP1 can be due to its ability to localize to a specific site on eMBP-1. Accordingly, binding to eMBP-1 will neutralize the toxic effects and alleviate the symptoms associated therewith. As disclosed herein, eMBP-1 may be targeted with a composition that localizes to tissues expressing eosinophil-related inflammation and has a therapeutic effect on the tissue by binding with eMBP-1. As used herein, “eMBP-1” and “MBP-1” refer to the same protein, mean the same and are used interchangeably.

Compositions Comprising High Molecular Weight Heparin or Unfractionated Heparin

Disclosed herein are compositions comprising heparin configured to be administered to a patient. In some aspects, the composition comprises high molecular weight heparin (HMWH) or a salt thereof (e.g., heparin sodium) and a pharmaceutically acceptable excipient. The HMWH can have a high purity, i.e., a substantial fraction of the heparin chains have a high molecular weight. In some aspects, the composition comprises unfractionated heparin (UFH) or a salt thereof (e.g., heparin sodium) and a pharmaceutically acceptable excipient.

In some aspects, the HMWH comprises an average molecular weight of about 20 kDa or greater. In some aspects, the HMWH can comprise an average molecular weight of 20 kDa, 21 kDa, 22 kDa, 23 kDa, 24 kDa, 25 kDa, 26 kDa, 27 kDa, 28 kDa, 29 kDa, 30 kDa, or individual values or ranges therebetween. It is additionally contemplated that the HMWH may have an average molecular weight above 30 kDa. In some aspects, the HMWH comprises an average molecular weight of about 35 kDa. In some aspects, the HMWH comprises an average molecular weight of about 40 kDa. In some aspects, the HMWH comprises an average molecular weight greater than 40 kDa. In some aspects, the average molecular weight of the HMWH is an individual value between the values disclosed herein or a range between values disclosed herein.

The average molecular weight of the HMWH may be selected to optimize binding to sites expressing eosinophilic inflammation. Because HMWH exhibits a higher affinity for eMBP-1 than low molecular weight heparin (LMWH) or unfractionated heparin (UFH), HMWH will bind more avidly than LMWH or UFH to sites of eosinophilic inflammation. In some aspects, a HMWH with a relatively high average molecular weight (e.g., 30 kDa) may bind more avidly than a HMWH with a relatively lower average molecular weight (e.g., 20 kDa). In some aspects, the binding affinity of the HMWH increases linearly with the average molecular weight of the HMWH. Accordingly, as the average molecular weight of the HMWH increases, the quantity of heparin required for localization of eosinophilic inflammation can be reduced with the expectation that a greater percentage of administered heparin will localize to the inflammation sites.

The purity of the HMWH can be defined by the amount of heparin chains having a molecular weight above a predetermined threshold. For example, the predetermined threshold may be 20 kDa and accordingly the purity of the HMWH can be determined based on a fraction, percentage, or ratio of heparin chains having a molecular weight of 20 kDa or greater compared to those having a molecular weight of less than 20 kDa. In some aspects, at least about 50% of the heparin chains in the HMWH can have a molecular weight of 20 kDa or greater, which may also be referred to as a purity of 50% (i.e., “high purity”). In some aspects, the total percentage of heparin chains in the HMWH having a molecular weight of 20 kDa or greater may be 60%, 70%, 80%, 90%, 95%, greater than 95%, or individual values or ranges therebetween. Accordingly, the composition of HMWH may be described as having 60% purity, 70% purity, 80% purity, 90% purity, 95% purity, greater than 95% purity, or individual values or ranges therebetween. In some aspects, the HMWH can also be defined by a maximum amount of molecular chains with a molecular weight below the predetermined threshold. For example, the HMWH can comprise a percentage of heparin chains with a molecular weight below 20 kDa at or below 50%, 40%, 30%, 25%, 20%, 15%, 10%, 5%, less than 5%, or individual values or ranges therebetween. In some aspects, the HMWH can also be defined by a maximum amount of molecular chains having a molecular weight below a cutoff defining low molecular weight chains (e.g., 8 kDa). For example, the HMWH can comprise a percentage of heparin chains with a molecular weight below 8 kDa at or below 50%, 40%, 30%, 25%, 20%, 15%, 10%, 5%, substantially 0%, or individual values or ranges therebetween.

In some aspects, a HMWH with a relatively high purity (e.g., 80%) can demonstrate greater localization to the eosinophil-related inflammation site than a HMWH with a lower purity (e.g., 50%). In some aspects, the localization rate of the HMWH increases as the purity of the HMWH increases. Accordingly, as the purity of the HMWH increases, the quantity of heparin required for adequate localization of eosinophilic inflammation can be reduced with the expectation that a greater percentage of administered heparin will localize to the inflammation sites.

In some aspects, the predetermined threshold for molecular weight that is used to define the “purity” of the HMWH can be a value other than 20 kDa. The predetermined threshold can be set based on the minimum desired average molecular weight for the HMWH composition. For example, the predetermined threshold for assessing purity of the HMWH can be 20 kDa, 21 kDa, 22 kDa, 23 kDa, 24 kDa, 25 kDa, 26 kDa, 27 kDa, 28 kDa, 29 kDa, 30 kDa, 35 kDa, 40 kDa, greater than 40 kDa, or individual values or ranges therebetween. Similarly, the cutoff of the low molecular weight chains can be a value other than 8 kDa. For example, the cutoff may be 5 kDa, 6 kDa, 7 kDa, 8 kDa, 9 kDa, 10 kDa, 11 kDa, 12 kDa, greater than 12 kDa, or individual values or ranges therebetween.

In a case where high purity is defined by a relatively higher threshold (e.g., 30 kDa), the HMWH can demonstrate greater localization to the eosinophil-related inflammation site than a case where high purity is defined by a relatively lower threshold (e.g., 20 kDa). In some aspects, the localization rate of the HMWH increases as the purity threshold increases. Accordingly, as the purity threshold of the HMWH increases, the quantity of heparin required for adequate localization of eosinophilic inflammation can be reduced with the expectation that a greater percentage of administered heparin will localize to the inflammation sites.

The compositions disclosed herein can comprise a specified quantity of HMWH heparin. In some aspects, the specified quantity of HMWH can be a dose of HMWH configured to reach or localize to an eosinophil-related inflammation site. In some aspects, the specified quantity of HMWH can be a therapeutically effective amount of HMWH. In some aspects, the specified quantity of HMWH can be a dose of HMWH configured to localize to the eosinophil-related inflammation site and facilitate imaging and/or diagnosis thereof. For example, where the eosinophil-related inflammation site is an esophagus or portion of an esophagus, the composition can comprises a quantity of HMWH selected from about 15000 units, about 10000 units, about 5000 units, about 4000 units, about 3000 units, about 2000 units, about 1000 units, about 500 units, about 250 units, less than about 250 units, or individual values or ranges therebetween. The quantity of HMWH may be about 100 mg, about 90 mg, about 80 mg, about 70 mg, about 60 mg, about 50 mg, about 40 mg, about 30 mg, about 20 mg, about 10 mg, about 5 mg, about 4 mg, about 3 mg, about 2 mg, about 1 mg, about 0.5 mg, less than about 0.5 mg, or individual values or ranges therebetween. In some aspects, the quantity of heparin can be diluted (e.g., with sterile saline) to provide a final volume of about 15 mL, 14 mL, 13, mL, 12, mL, 11 mL, 10 mL, about 9 mL, about 8 mL, about 7 mL, about 6 mL, about 5 mL, about 4 mL, about 3 mL, about 2 mL, about 1 mL, about 0.9 mL, about 0.8 mL, about 0.7 mL, about 0.6 mL, about 0.5 mL, about 0.4 mL, about 0.3 mL, about 0.2 mL, about 0.1 mL, less than about 0.1 mL, or individual values or ranges therebetween. The dose of HMWH may vary based on the size of the targeted eosinophil-related inflammation site. A larger quantity of HMWH can be required for targeting larger sites and/or organs. Where the eosinophil-related inflammation site is a different site or organ other than the esophagus as further described herein, the quantity of HMWH can be a value described herein or a larger or small value necessary to adequately target the eosinophil-related inflammation site as would be apparent to one having an ordinary level of skill in the art.

As described herein, the compositions generally comprise a relatively small quantity of HMWH heparin or a salt thereof because the high affinity for eMBP-1 and high purity of the composition results in a lower required dose as compared to LMWH or UFH. In some aspects, the amount of HMWH in the disclosed compositions and methods can be 60%, 50%, 40%, 30%, 20% or 10% than the amount of LMWH or UFH needed for the same result. Accordingly, the small quantity of HMWH poses a relatively low risk of HIT because the total quantity of heparin administered is low compared to commonly acceptable doses of LMWH or UFH. Further, even LMWH and UFH commonly include a quantity of high molecular weight chains due to their low purity (i.e., high polydispersity). Accordingly, in some cases the total quantity of high molecular weight chains in the composition can be substantially similar to the total quantity of high molecular weight chains found in typically acceptable doses of LMWH or UFH, and thus do not pose a substantially greater risk of HIT. Further, when HMWH is administered orally as described herein, the risk of HIT can be greatly diminished in comparison to the degree of risk typically associated with administration of heparin intravenously and/or subcutaneously.

In some aspects, the compositions can comprise unfractionated heparin. In some aspects, the unfractionated heparin can be heparin sodium. In some aspects, the heparin sodium can be 1000 USP units, 5000 USP units, 10,000 UPS units or any amount in between.

In some aspects, the dose or dosing regimen used in the methods disclosed herein can be the dose or dosing regiman described in Ashoor T M, et at, Nebulized heparin and salbutamol versus Salbutamol alone in acute exacerbation of chronic obstructive pulmonary disease requiring mechanical ventilation: a double blind randomised controlled trial, Korean J Anesthesiol. 2020 Feb. 28 or Hiremath M, et al., Heparin in the long-term management of ligneous conjunctivitis: a case report and review of literature, Blood Coagul Fibrinolysis. 2011 October; 22(7):606-9.

In some aspects, the compositions disclosed herein are administered orally. For example, the composition can be swallowed orally by the subject. In some aspects, the composition can be administered orally with a syringe, dropper, or other device.

In some aspects, the compositions disclosed herein can be administered orally or topically as an oral or topical solution. For example, compositions comprising UFH or HMWH can be formulated as an oral solution or a topical solution for treating eosinophilic GI disorders (EGIDs), including by not limited to EoE and eosinophilic gastroenteritis; and inflammatory bowel disease, including by not limited to ulcerative colitis and Crohn's disease.

In some aspects, the compositions disclosed herein can be administered by inhalation as a nasal spray. For example, compositions comprising UFH or HMWH can be formulated as a nasal spray for treating eosinophilic chronic rhinosinusitis or nasal polyps.

In some aspects, the compositions disclosed herein can be administered topically (e.g., eye drops). For example, compositions comprising UFH or HMWH can be formulated for topical administration for treating ocular diseases having an allergic pathophysiological component including but not limited to eosinophilic conjunctivitis, seasonal and/or perennial allergic conjunctivitis, vernal conjunctivitis, atopic keratoconjunctivitis, giant papillary conjunctivitis or contact dermatoconjunctivitis.

The compositions disclosed herein can be configured or formulated for additional administration routes. For example, where the composition is administered to treat other eosinophil-related conditions and diseases than EoE, different administration routes may be necessary or preferable. In some aspects, the compositions disclosed herein are configured for administration intravenously, topically, by inhalation and/or orally to treat gastrointestinal eosinophil-associated diseases. In some aspects, the gastrointestinal eosinophil-associated diseases that can be treated by oral (or topical) administration comprise EoE, eosinophilic gastritis, and/or eosinophilic gastroenteritis. In some aspects, the compositions disclosed herein are configured for administration by inhalation to treat inflammation in the nose, paranasal sinuses and lung. In some aspects, the compositions disclosed herein are configured for administration by an enema to treat the colon. In some aspects, the compositions disclosed herein are configured for administration by catheter to treat eosinophil-related inflammation in the urinary bladder. In some aspects, the compositions disclosed herein are configured for administration by eye drops to treat ocular eosinophilic-related inflammation or diseases having an allergic pathophysiological component. In some aspects, the compositions disclosed herein are configured for topical administration as a cream or ointment to treat eosinophil-related inflammation and/or diseases of the skin.

While the composition is substantially described in regards to administration to an esophagus, the composition may be configured for administration to additional tissues or organs. In some aspects, the targeted eosinophil-related inflammation or eosinophilic disease may be specific to the gastrointestinal tract (e.g., mouth, esophagus, stomach, small intestine, large intestine, or colon), lung, nose, eye, skin, one or more joints, one or more muscles, one or more nerves, heart, kidney, bladder, uterus, prostate, breast, lymph or blood.

In some aspects, the compositions can further comprise one or more additional agents. In some aspects, the compositions can further comprise a tracer such as a radiolabeled contrast agent conjugated to the HMWH. For example, the radiolabeled contrast agent can be 99mTc. However, other tracers, such as tracers used for positron emission tomography, can also be employed for detecting the binding of the high molecular weight heparin to sites of eosinophilic inflammation. In some aspects, the tracers can be any tracer or label in Table 1. Accordingly, when the composition is administered as described herein, conventional imaging modalities (e.g., X-ray) may be used to visualize the eosinophil-related inflammation and/or disease. For example, in the case of EoE, the composition can be administered to facilitate visualization of the entire esophagus.

In some aspects, the tracer can be used to diagnose eosinophil-related inflammation and/or disease. For example, the composition comprising a tracer (i.e., diagnostic agent) may be administered as described herein and conventional imaging modalities (e.g., X-ray) may be used to capture one or more images of the patient. Localization of the HMWH can be assessed based on the location and concentration of the detected tracer in the one or more images. Accordingly, the patient may be diagnosed with eosinophil-related inflammation and/or disease based on the one or more images. In some aspects, at least one first image of the patient is acquired at a first time and at least one second image of the patient is acquired at a second time. The first image and the second image can be compared to monitor and assess progression of the inflammation and/or disease activity. In some aspects, additional images may be acquired at additional times to continue to monitor and assess the patient. In some aspects, a separate administration of the composition can occur prior to acquiring each of the first image, the second image, and any of the additional images. However, in some aspects, a single administration of the composition can provide adequate radiolabeling for more than one set of images. The composition can be utilized for monitoring and assessing any of the eosinophil-related conditions and diseases described herein with respect to treatment.

In some aspects, the compositions can further comprise a therapeutic agent conjugated to the HMWH. In some aspects, the compositions further comprise a therapeutically effective amount of a therapeutic agent for administration to the patient. In some aspects, the therapeutic agent is configured (or formulated) to have a therapeutic effect on the eosinophil-related inflammation and/or disease. As disclosed herein, by conjugating therapeutic agents to HMWH, a treatment can be targeted directly to an area(s) of inflammation because the avidity of the HMWH for tissue bound eMBP-1. Thus, the targeting of the HMWH conjugated to a therapeutic agent (e.g., a HMWH/therapeutic agent complex) directly to one or more sites of eosinophil-related inflammation can reduce the quantity (or dose) of the therapeutic agent needed for care, and thus limit or minimize any side effects associated with the administration of the therapeutic agent. Accordingly, the therapeutically effective amount of the therapeutic agent can be less than a therapeutically effective amount typically associated with administration of the therapeutic agent in the absence of HMWH or another targeted mechanism. In some aspects, the therapeutic agent is a glucocorticoid, which is an effective treatment for eosinophil-related diseases. In some aspects, the glucocorticoid is one or more of mometasone, fluticasone, budesonide, and methylprednisolone. Additional therapeutic agents for eosinophil-related inflammation or diseases are contemplated as would be apparent to one having an ordinary level of skill in the art.

The compositions disclosed herein can further comprise various additional components or additives as would be known to a person having an ordinary level of skill in the art. In some aspects, the compositions further comprise stannous chloride. In some aspects, the compositions further comprise a stabilizing agent. In some aspects, the compositions further comprise a taste-masking agent.

Compositions Comprising a Radiolabeled Contrast Agent

Disclosed herein are compositions comprising a radiolabeled contrast agent. In some aspects, the radiolabeled contrast agent comprises heparin. In some aspects, heparin can be high molecular weight heparin. In some aspects, heparin can be low molecular weight heparin. In some aspects, the radiolabel can be ^{99m}Tc. In some aspects, the radiolabeled heparin comprises heparin having an average molecular weight from about 20 kDa to about 40 kDa, wherein at least 50% of heparin chains in the heparin have a molecular weight of at least 20 kDa.

In some aspects, a radiolabeled contrast agent or any of the compositions disclosed herein comprising a radiolabeled contrast agent, for example, ^{99m}Tc-heparin, can be administered to a subject orally or by intravenous injection. In some aspects, in any of the methods described herein, the method of administration of a radiolabeled contrast agent, for example, 99mTc-heparin, to a subject can be oral. Oral dosing can entail ingestion similar to routine barium studies of the esophagus. A radiolabeled contrast agent can be suspended in a thickened mixture (i.e., sucralose). Examples of thickening agents include, but are not limited to, dietary starches, such as agar-agar, alginate, carrageenan, cassia gum, cellulose gum, gellan gum, guar gum, hydroxypropylcellulose, konjac gum, locust bean gum, methylcellulose, hydroxypropyl methylcellulose, microcrystalline cellulose, pectin, and xanthan gum. Other viscosifiers include honey, agave nectar, date nectar, Kuzu or Kudzu root, arrow root, corn syrup, thick juices, maple syrup, coconut oil, and palm oil.

Disclosed herein are methods of preparing radiolabeled heparin (e.g. Tc-99m-Heparin). In some aspects, the methods can comprise preparing 20 mg/mL stannous chloride dihydrate in sterile water under flowing medical-grade nitrogen. In some aspects, the methods can further comprise filtering 0.3 mL solution through a 0.22 micro filter and mixing it with 1-150 mg of low molecular weight heparin sodium. In some aspects, the methods can further comprise filtering 0.3 mL solution through a 0.22 micro filter and mixing with 1-150 mg of high molecular weight heparin sodium. In some aspects, Tc99m-heparin can be prepared on the day of imaging. In some aspects, the methods can further comprise calibrating Tc-99m. In some aspects, Tc-99m can be calibrated for a time of patient administration. In some aspects, the calibrating step can further comprise eluting the Tc-99m in 0.4 mL using a Tc-99m generator. In some aspects, the methods can further comprise adding a heparin solution to the calibrated Tc-99m to and incubating the Tc-99m-heparin solution (e.g., radiolabeled solution). In some aspects, the incubating step can be about 5 minutes at 20° C. In some aspects, the Tc-99m-heparin solution (e.g., radiolabeled solution) can be prepared for oral administration. In some aspects, the Tc-99m-heparin solution (e.g., radiolabeled solution) can be diluted in sterile saline. In some aspects, the Tc-99m-heparin solution (e.g., radiolabeled solution) can be diluted in sterile saline for a final volume of 1, 5, 10, or 15 mL. In some aspects, the Tc-99m-heparin solution (e.g., radiolabeled solution) can be diluted in sterile saline for a final volume of 15 mL. In some aspects, the radiolabeled heparin comprises heparin having an average molecular weight from about 20 kDa to about 40 kDa, wherein at least 50% of heparin chains in the heparin have a molecular weight of at least 20 kDa.

In some aspects, heparin can be low molecular weight heparin. In some aspects, heparin can be high molecular weight heparin. As such, radiolabeled heparin can include radiolabeled high molecular weight heparin or radiolabeled low molecular weight heparin. In some aspects, the radiolabeled heparin comprises heparin having an average molecular weight from about 20 kDa to about 40 kDa, wherein at least 50% of heparin chains in the heparin have a molecular weight of at least 20 kDa.

The radiolabeled heparin disclosed herein can be prepared at various doses. For example, the radiolabeled heparin can be 0.1, 0.2, 0.3, 0.4, 0.5, 0.6, 0.7, 0.8, 0.9, 1.0, 1.5, 2.0, 2.5, 3, 3.5, 4.0, 4.5, 5.0, 5.5, 6.0, 6.5, 7.0, 7.5, 8.0, 8.5, 9.0, 9.5, or 10.0 mCi. In some aspects, the dose of radiolabeled heparin can be 0.3 mCi to around 1 mCi. In some aspects, the dose of radiolabeled heparin can be 1.0 mCi. In some aspects, the dose of radiolabeled heparin can be 10 mCi. In some aspect, the radiolabeled heparin can be Tc-99m-heparin. In some aspects, the doses of Tc-99m-heparin can be 0.1, 0.2, 0.3, 0.4, 0.5, 0.6, 0.7, 0.8, 0.9, 1.0, 1.5, 2.0, 2.5, 3, 3.5, 4.0, 4.5, 5.0, 5.5, 6.0, 6.5, 7.0, 7.5, 8.0, 8.5, 9.0, 9.5, or 10.0 mCi. In some aspects, the dose of Tc-99m-heparin can be 0.3 mCi to 1 around mCi. In some aspects, the dose of Tc-99m-heparin can be 1.0 mCi. In some aspects, the dose of Tc-99m-heparin can be 10 mCi. In some aspects, 1-100 mg of USP heparin can be labeled with 0.1 to 30 mCi of a radiolabel (e.g., Tc-99m). In some aspects, about 88 mg of USP heparin can be labeled with about 30 mCi of a radiolabel (e.g., Tc-99m). In some aspects, about 0.1 to about 1 mg of heparin can be used to label with a radiolabel (e.g., Tc-99m).

In some aspects, the dose of heparin used to bind to Tc-99m can change or vary depending on the form or type of heparin. In some aspects, using high molecular weight heparin allows for the use of smaller amounts or doses of a radiolabel because it results in a higher rate of uptake in, for example, the esophageal tissue, compared to unfractionated heparin. In some aspects, the 1 mg to 88 mg of unfractionated (or low molecular weight) heparin can be labeled with 0.3 to 30 mCi Tc-99m.

In some aspects, when the heparin used is high molecular weight heparin, the dose or amount of the high molecular weight heparin can be less, compared to the amount of unfractionated heparin that would be needed, used or required. In some aspects, the amount of high molecular weight heparin can be 0.1 to about 1 mg, 1 mg to 2 mg, or 2 mg to 3 mg to label with a radiolabel. In some aspects, 1 mg of high molecular weight heparin can be used to label with a radiolabel and bind better than 3 mg of unfractionated heparin. In some aspects, the 0.1 mg to 88 mg of high molecular weight heparin can be labeled with 0.3 to 30 mCi Tc-99m.

In some aspects, lower amounts of HMWH can be used to bind to eMBP1 in tissues compared to UFH or LMWH. For example, using 1 mg HMWH can generate a better image than 3 mg UFH. Further, using 3 mg UFH and 3 mCi binds results in a tiny fraction of the heparin being labeled (1 Tc per 18,000 heparin molecules). By reducing the heparin (e.g., HMWH) to 1 mg, the ratio of heparin to radiolabel would be increased to about 1 Tc per 6000 heparin molecules or less). Thus, the heparin with a higher specific activity would yield a better image because less cold heparin is competing with the hot heparin for binding to eMBP1.

In some aspects, the compositions disclosed herein can be administered intravenously, topically and/or orally (e.g., by swallowing the compositions disclosed herein) to identify gastrointestinal eosinophil-associated diseases. In some aspects, the gastrointestinal eosinophil-associated diseases that can be detected after oral administration include but are not limited to eosinophilic esophagitis, eosinophilic gastritis, hypereosinophilic syndrome, and eosinophilic gastroenteritis. In some aspects, administration of the compositions disclosed herein by inhalation can be used to identify inflammation in the nose, paranasal sinuses and lung. In some aspects, administration of the compositions disclosed herein by intravenous can be used to identify inflammation in the heart. In some aspects, administration of the compositions disclosed herein by an enema can be used to investigate the colon. In some aspects, administration of the compositions disclosed herein by catheter can be used for identifying eosinophil-related inflammation in the urinary bladder. In some aspects, administration of the compositions disclosed herein by eyedrops can be used for identifying of ocular eosinophilic-related inflammation.

Disclosed herein are method that include, in part, administering radiolabeled heparin to a subject. In some aspects, the radiolabeled heparin comprises heparin having an average molecular weight from about 20 kDa to about 40 kDa, wherein at least 50% of heparin chains in the heparin have a molecular weight of at least 20 kDa. In some aspects, the radiolabeled heparin is administered orally to a subject. In some aspects, the dwell time of the radiolabeled heparin in the esophagus can be controlled by varying the viscosity of a contrast agent and/or by increasing the time interval between swallows, thereby providing more time for a contrast agent to contact and bind to an eosinophil granule protein. Further, having a subject lie down with head below feet, so that there is some reflux within the esophagus, can prolong contact between a contrast agent and the mucosal tissue of the esophagus in a subject.

In some aspects, the radiolabeled contrast agent (e.g., radiolabeled heparin such as 99mTc-heparin) can be administered orally over 15 minutes. In some aspects, the radiolabeled contrast agent (e.g., radiolabeled heparin such as 99mTc-heparin) can be administered orally over 15 minutes in 1 ml aliquots (e.g., 1 ml/minute). In some aspects, 15 ml of a radiolabeled contrast agent can be administered (e.g., swallowed orally by the subject). In some aspects, the subject can perform 15 swallows of 1 ml of the radiolabeled contrast agent. In some aspects, the number of swallows of the radiolabeled contrast agent can be 1, 2, 3, 4, 5, 6, 7, 8, 9, 10, 11, 12, 13, 14, or 15. In some aspects, the 1 ml aliquots of the radiolabeled contrast agent (e.g., 99mTc-heparin) can be administered to a subject with a syringe while the subject is in the supine position. In some aspects, the subject can remain in the supine position for at least 1, 5, 10, 15, 20, 25, 30 minutes or any number in between. In some aspects, the subject can swallow 1, 2, 3, 4, 5, 10, 15, 20, 25, 30, 35, 40, 45, 50, 55, 65, 70, 75, 80, 85, 90, 95, 100 ml, or any amount in between after remaining in the supine position. In some aspects, the subject can swallow 100 ml of water after remaining in the supine position for at least 15 minutes. In some aspects, the methods can further comprise administering water after administration of the radiolabeled contrast agents to remove weakly bound heparin. Water can be administered after each administration of the radiolabeled contrast agent or after all of the radiolabeled contrast agent is administered to the subject. In some aspects, the subject can swallow 1, 5, 10, 15, 20, 25, 30, 35, 40, 45, 50, 55, 65, 70, 75, 80, 85, 90, 95, 100 ml, or any amount in between, of water after each administration of the radiolabeled contrast agent or after all of the radiolabeled contrast agent. In some aspects, the subject can swallow 100 ml of water with 15 swallows of approximately 7 ml of water. In some aspects, the first swallow of water can occur at about 15 to 30 minutes after the last swallow of the radiolabeled contrast agent.

In some aspects, a radiolabeled contrast agent can be administered to a subject in a volume from about 0.5 mL to about 1,000 mL, including all volumes in between 0.5 mL and 1,000 mL. A person of skill can determine by methods well known in the art the volume of a radiolabeled contrast agent to be administered to a subject based on the age, sex, weight, and overall condition of a subject. For example, in some aspects, the volume of a radiolabeled contrast agent administered to a subject can be from about 0.5 mL to about 5 mL. In some aspects, the volume of a radiolabeled contrast agent administered to a subject can be from about 5 mL to about 250 mL. In some aspects, the volume of a radiolabeled contrast agent administered to a subject can be from about 10 mL to about 125 mL. In some aspects, the volume of a radiolabeled contrast agent administered to a subject can be from about 15 mL to about 100 mL. Thus, the volume of a radiolabeled contrast agent that can be administered to a subject can be, for example, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mL, and all volumes in between. In some aspects, the radiolabeled contrast agent can be ^{99m}Tc-heparin. In some aspects, the radiolabeled heparin can be radiolabeled unfractionated heparin. In some aspects, the radiolabeled heparin can be radiolabeled high molecular weight heparin. In some aspects, the radiolabeled heparin can be radiolabeled low molecular weight heparin. In some aspects, the compositions comprising radiolabeled heparin can be used in any of the methods disclosed herein. In some aspects, the compositions comprising radiolabeled heparin can be used to detect eMBP-1. In some aspects, the compositions comprising radiolabeled heparin can be used to bind to eMBP-1. In some aspects, the radiolabeled heparin in the methods disclosed herein can be a high molecular weight heparin.

High molecular weight heparin. Heparin is made up of long-chain glycosaminoglycans, and can have a diverse molecular weight consisting of a variable number of sulphated repeating disaccharide units. Disclosed herein are compositions comprising high molecular weight heparin (e.g., estimated as 20-30 kDa). In some aspects, compositions comprising high molecular weight heparin may be a more effective reagent for the localization of eosinophil-associated inflammation than unfractionated heparin. In some aspects, compositions comprising high molecular weight heparin can be used to treat eosinophil-associated inflammation, and may be more effective than unfractionated heparin.

Described herein is the finding that high molecular weight heparin, estimated as 20-30 kDa, binds surprisingly more avidly to the eosinophil granule eMBP-1 than other lower molecular weight forms of heparin. The ability of compositions comprising high molecular weight heparin to bind with a higher affinity for eMBP-1 than other lower molecular weight forms of heparin can permit using less heparin than previously used for localization of eosinophilic inflammation, and, allow said compositions comprising high molecular weight heparin to be used to treat one or more eosinophil-associated diseases. Although studies disclosed herein label heparin with technetium-99M, other tracers, such as tracers used for positron emission tomography, can also be employed for detecting the binding of the high molecular weight heparin to sites of eosinophilic inflammation. In some aspects, the tracers can be any tracer or label in Table 1.

In some aspects, the eosinophil-related inflammation or eosinophilic disease can be tissue- or organ-specific. In some aspects, the eosinophil-related inflammation or eosinophilic disease can be specific for the gastrointestinal tract, lung, nose, eye, skin, one or more joints, one or more muscles, one or more nerves, heart, kidney, bladder, uterus, prostate, breast, lymph or blood.

In some aspects, the eosinophil-related inflammation or eosinophilic disease can be eosinophilic gastrointestinal disorders. Examples of eosinophilic gastrointestinal disorders include but are not limited to eosinophilic esophagitis, eosinophilic gastritis, eosinophilic gastroenteritis, eosinophilic enteritis, eosinophilic cholecystitis, and eosinophilic colitis. eosinophil-related inflammation can be inflammatory bowel disease including ulcerative colitis or Crohn's disease. In some aspects, the eosinophil-related inflammation or eosinophilic disease can be an eosinophilic pancreatitis. In some aspects, the eosinophil-related inflammation or eosinophilic disease can be an eosinophilic hepatitis. In some aspects, the eosinophil-related inflammation or eosinophilic disease can be an eosinophilic ascites. In some aspects, the eosinophil-related inflammation or eosinophilic disease can be a pulmonary eosinophilic syndrome. Examples of a pulmonary eosinophilic syndrome include but are not limited to eosinophilic asthma, eosinophilic bronchitis, eosinophilic pneumonia, and eosinophil pleuritis. In some aspects, the eosinophil-related inflammation or eosinophilic disease can be an eosinophilic myocarditis. In some aspects, the eosinophil-related inflammation or eosinophilic disease can be eosinophilic coronary arteritis. In some aspects, the eosinophil-related inflammation or eosinophilic disease can be eosinophilic rhinosinusitis. In some aspects, the eosinophil-related inflammation or eosinophilic disease can be eosinophilic nasal polyposis. In some aspects, the eosinophil-related inflammation or eosinophilic disease can be an eosinophilic ocular disorder. Examples of eosinophilic ocular disorder include but are not limited to allergic conjunctivitis (e.g., seasonal and perennial), giant papillary conjunctivitis, and keratoconjunctivitis (atopic and vernal)). In some aspects, the eosinophil-related inflammation or eosinophilic disease can be eosinophilic conjunctivitis, vernal conjunctivas or contact dermatoconjunctivitis. In some aspects, the eosinophil-related inflammation or eosinophilic disease can be an eosinophilic nephritis. In some aspects, the eosinophil-related inflammation or eosinophilic disease can be an eosinophilic cystitis. In some aspects, the eosinophil-related inflammation or eosinophilic disease can be an eosinophilic prostatitis. In some aspects, the eosinophil-related inflammation or eosinophilic disease can be an eosinophilic endometritis. In some aspects, the eosinophil-related inflammation or eosinophilic disease can be an eosinophilic myometritis (uterus). In some aspects, the eosinophil-related inflammation or eosinophilic disease can be an eosinophilic mastitis. In some aspects, the eosinophil-related inflammation or eosinophilic disease can be an eosinophil-related neuropathy. In some aspects, the eosinophil-related inflammation or eosinophilic disease can be an eosinophilic synovitis. In some aspects, the eosinophil-related inflammation or eosinophilic disease can be an eosinophilic myositis. In some aspects, the eosinophil-related inflammation or eosinophilic disease can be an eosinophilic panniculitis. In some aspects, the eosinophil-related inflammation or eosinophilic disease can be an eosinophilic fasciitis (Shulman syndrome). In some aspects, the eosinophil-related inflammation or eosinophilic disease can be chronic rhinosinusitis or a nasal polyp.

In some aspects, the eosinophilic disease can be eosinophilic cystitis, eosinophilic fasciitis, eosinophilic colitis, eosinophilic esophagitis, eosinophilic gastritis, eosinophilic gastroenteritis, eosinophilic granulomatosis with polyangiitis, eosinophilic pneumonia, hypereosinophilic syndrome, vernal conjunctivitis, giant papillary conjunctivitis, atopic dermatitis, chronic rhinosinusitis or transplant rejection.

In some aspects, the eosinophil-related inflammation can be caused by a parasitic disease; an allergic reaction; asthma; an autoimmune disease; a drug reaction; an environmental exposure; a topical contact; a genetic disease; a transplant rejection, a hematologic or lymphocytic disease, or an inflammatory or immunological reaction with expression of eosinophil differentiation, chemoattracting, activating factors or a combination thereof. Examples of a parasitic disease can include but is not limited to helminthic infections and ectoparasites. Examples of drug reactions include but are not limited to drug hypersensitivity reactions (e.g., drug reactions with eosinophilia and systemic symptoms (DRESS) with potential for prolonged sequelae). In some aspects, the eosinophil-related inflammation can be caused by a solid tumor (e.g., a malignancy), a lymphoma or a leukemia. In some aspects, the activating factor can be a marker for a cancer. In some aspects, eosinophils can indicate a gastrointestinal cancer.

In some aspects, the eosinophil-related inflammation or eosinophilic disease can be eosinophil-related syndrome.

In some aspects, eosinophil-related syndromes can include eosinophilia myalgia syndrome (EMS) and toxic oil syndrome (TOS). Eosinophilia myalgia syndrome and toxic oil syndrome include but are not limited to severe myalgia plus hypereosinophilia (peripheral blood and/or tissue) or eosinophilia, often accompanied by neurologic symptoms and skin changes. Epidemic cases of EMS have been attributed to contaminated L-tryptophan exposure. Epidemic cases of TOS have been attributed to rapeseed oil denatured with aniline.

In some aspects, eosinophil-related syndromes can include eosinophilic granulomatosis with polyangiitis (Churg-Strauss syndrome). Symptoms of eosinophilic granulomatosis with polyangiitis (Churg-Strauss syndrome) include but are not limited necrotizing vasculitis with hypereosinophilia; antineutrophil cytoplasmic antibodies (e.g., ANCA1 and ANCA2 subvariants); 4 of 6 criteria including asthma, eosinophilia, history of allergy, nonfixed pulmonary infiltrates, paranasal sinus abnormalities, and extravascular eosinophils.

In some aspects, eosinophil-related syndromes can include episodic angioedema with eosinophilia (Gleich syndrome). Episodic angioedema with eosinophilia (Gleich syndrome) can include but is not limited to cyclic recurrent angioedema, hypereosinophilia, and increased IgM levels, often with clonal T cells, one of several possible clinical presentations of secondary/reactive hypereosinophilic syndromes). Hypereosinophilic syndromes can include peripheral blood hypereosinophilia, hypereosinophilia-related organ damage.

In some aspects, eosinophil-related syndromes can include hyper-IgE syndromes. Hyper-IgE syndromes can include but are not limited to hereditary immunodeficiency syndromes with hypereosinophilia and increased IgE levels, often with eczema and facial anomalies; and known gene mutations: autosomal dominant hyper-IgE syndrome, signal transducer and activator of transcription 3 (STAT3) mutations and autosomal recessive hyper-IgE syndrome, dedicator of cytokinesis 8 (DOCKS) mutations.

In some aspects, eosinophil-related syndromes can include IgG4-related diseases. IgG4-related diseases include but are not limited to a spectrum of disorders with fibrosis as a major finding, tumor-like swelling of tissues and organs, tissue eosinophilia, and increased IgG4.

In some aspects, eosinophil-related syndromes can include Omenn syndrome. Omenn syndrome includes but is not limited to severe combined immunodeficiency with hypereosinophilia, often with erythroderma, hepatosplenomegaly, and lymphadenopathy and autosomal recessive genetic disease (recurrent mutations in recombination-activating gene (e.g., RAG1 or RAG2).

In some aspects, the eosinophil-related inflammation or eosinophilic disease can be an eosinophil-related dermatoses. Disclosed herein are different and overlapping compartments associated with a variety of diseases, including but not limited to: epidermis (e.g., eosinophilic spongiosis); dermis, connective tissue (e.g., eosinophilic cellulitis); dermis, blood vessels (e.g., eosinophilic vasculitis; hair follicles (e.g., eosinophilic folliculitis); subcutaneous fat (e.g., eosinophilic panniculitis); fascia (e.g., eosinophilic fasciitis); muscle (e.g., eosinophilic myositis); and nerve (e.g., eosinophilic neuritis).

In some aspects, the eosinophil-related inflammation or eosinophilic disease can be allergic contact dermatitis; angiolymphoid hyperplasia with eosinophilia; annular erythema of infancy; atopic dermatitis; bullous pemphigoid and pemphigoid variants; coccidiomycosis; drug eruptions; eosinophilic fasciitis; eosinophilic, polymorphic, and pruritic eruption associated with radiotherapy; eosinophilic pustular folliculitis: all variants; erythema toxicum neonatorum; eosinophilic ulcer of the oral mucosa; eosinophilic vasculitis; granuloma faciale; infestations (parasites/ectoparasites, including scabies, bed bugs, and cutaneous larva migrans); incontinentia pigmenti; kimura disease; langerhans cell histiocytosis; mycosis fungoides and Sezary syndrome/cutaneous lymphoma; pachydermatous eosinophilic dermatitis; pemphigoid variants, including bullous pemphigoid and pemphigoid gestationis; pemphigus variants; pregnancy-related dermatoses; pseudolymphoma; urticaria/angioedema; vasculitis; or Wells syndrome (various diseases with eosinophilic cellulitis). See Starr J, et al, Mayo Clin Proc 75:755-759, 2000; incorporated herein by reference.

Methods of Producing Medical Images

In some aspects, the compositions disclosed herein can be configured for imaging of the eosinophil-related inflammation. For example, the disclosed compositions can comprise radiolabeled heparin. As disclosed herein, the compositions can comprise a tracer such as a radiolabeled contrast agent conjugated to HMWH. In some aspects, the radiolabeled contrast agent may be ^{99m}Tc.

Disclosed herein are methods of producing a medical image of a tissue, organ, or body part or a combination thereof using radiolabeled heparin. Disclosed herein are methods of producing a medical image of one or more of a tissue, organ, or body part or a combination thereof Also disclosed herein are methods of diagnosing eosinophil-related inflammation in a subject. Further disclosed herein are methods of detecting eosinophil degranulation in a subject. In some aspects, the radiolabeled heparin comprises a tracer such as a radiolabeled contrast agent conjugated to HMWH.

Also disclosed herein are methods of producing a medical image of an organ in a subject. In some aspects, the methods can comprise detecting an eosinophil granule protein in the mucosal tissue of the organ in a subject. In some aspects, the methods can comprise administering to a subject radiolabeled heparin (e.g., radiolabeled high molecular weight heparin) under conditions wherein the radiolabeled heparin binds to an eosinophil granule protein to form a radiolabeled heparin/eosinophil granule protein complex. In some aspects, the methods can comprise detecting the radiolabeled heparin/eosinophil granule protein complex in the mucosal tissue of the organ. In some aspects, detecting the radiolabeled heparin/eosinophil granule protein complex in the mucosal tissue of the organ can produce a medical image of the organ in the subject. In some aspects, the heparin has an average molecular weight from about 20 kDa to about 40 kDa, wherein at least 50% of heparin chains in the heparin have a molecular weight of at least 20 kDa.

Further disclosed herein are methods of imaging a tissue exhibiting eosinophil-related inflammation in a subject. In some aspects, the method comprises administering a composition comprising an effective amount of radiolabeled high molecular weight heparin (or a salt thereof) and a pharmaceutically acceptable excipient to the subject, wherein the high molecular weight heparin binds to one or more eosinophil granule proteins in the tissue. The method further comprises detecting the radiolabeled high molecular weight heparin to produce a medical image of the tissue. In some aspects, detecting the radiolabeled high molecular weight heparin comprises detecting a complex from the radiolabeled heparin binding to the eosinophil granule proteins. The high molecular weight heparin can have a high purity, i.e., a substantial fraction of the heparin chains have a high molecular weight. In some aspects, the heparin is labeled with ^{99m}Tc or another radiolabeled contrast agent or tracer as described herein. In some aspects, the medical image comprises a furrow, a ring, and/or a stricture along an esophagus (i.e., a symptom of EoE).

In some aspects, methods can include administering to a subject a composition comprising one or more radiolabeled contrast agents. In some aspects, the methods can include administering to a subject a composition comprising one or more radiolabeled contrast agents to enhance the detection of eosinophil granule proteins in the mucosal tissue in a tissue, organ or body part of a subject. In some aspects, the composition can be any of the compositions disclosed herein, for example radiolabeled heparin. In some aspects, the compositions can comprise a radiolabeled contrast agent. In some aspects, the composition can comprise ^{99m}Tc-heparin, heparin, or ¹⁴C-heparin, or any combination thereof. In some aspects, a radiolabeled contrast agent can be ^{99m}mTc-heparin. Examples of radiolabeled heparin/eosinophil granule protein complexes include, but are not limited to, ^{99m}Tc-heparin/MBP-1, ^{99m}Tc-heparin/MBP, ^{99m}Tc-heparin/MBP-2, ^{99m}Tc-heparin/EDN, ^{99m}Tc-heparin/ECP, and ^{99m}Tc-heparin/EPO.

In some aspects, after administering to a subject a composition comprising a radiolabeled contrast agent, for example ^{99m}Tc-heparin, the methods can further comprise using one or more technologies and/or processes to detect the radiolabeled contrast agent/eosinophil granule protein complexes in a tissue, organ or body part (e.g., the mucosal tissue of the esophagus) in a subject. In some aspects, the heparin has an average molecular weight from about 20 kDa to about 40 kDa, wherein at least 50% of heparin chains in the heparin have a molecular weight of at least 20 kDa. In some aspects, the one or more eosinophils may have degranulated and caused one or more patches of inflammation, creating a medical image to map the distribution of inflammation and/or deposition of eosinophil granule proteins. These images can be used to detect and/or to study the anatomy and/or pathophysiology of eosinophilic esophagitis. Examples of technologies that can be used to create a medical image include, but are not limited to, single photon emission computed tomography (SPECT), positron emission (PET) scans, X-ray, conventional or computed tomography (CT), a combination of SPECT and CT, or magnetic resonance imaging (MRI). In some aspects, for example, SPECT can optionally be used in combination with MRI and/or CT scans to produce a medical image of an esophagus having patches of eosinophilic esophagitis. Fiduciary markers on the skin of a subject can also be used to position a subject so that the subject can be imaged from day to day. For example, lasers can be used to position a subject reproducibly. This permits use of multiple scans to be precisely compared. In some aspects, a medical image can be three-dimensional. In some aspects, a medical image can be two-dimensional.

In some aspects, when the composition is administered as described herein, conventional imaging modalities (e.g., X-ray) may be used to visualize the eosinophil-related inflammation and/or disease. For example, in the case of EoE, the composition can be administered to facilitate visualization of the entire esophagus. In some aspects, the tracer can be used to diagnose eosinophil-related inflammation and/or disease. For example, the composition comprising a tracer (i.e., diagnostic agent) can be administered as described herein and conventional imaging modalities (e.g., X-ray) may be used to capture one or more images of the patient. Localization of the HMWH can be assessed based on the location and concentration of the detected tracer in the one or more images. Accordingly, the patient can be diagnosed with respect to the eosinophil-related inflammation and/or disease based on the one or more images. In some aspects, at least one first image of the patient is acquired at a first time and at least one second image of the patient is acquired at a second time. The first image and the second image can be compared to monitor and assess progression of the inflammation and/or disease activity. In some aspects, additional images can be acquired at additional times to continue to monitor and assess the patient. In some aspects, a separate administration of the composition occurs prior to acquiring each of the first image, the second image, and the additional images. However, in some aspects, a single administration of the composition can provide adequate radiolabeling for more than one set of images. The composition can be utilized for monitoring and assessing any of the eosinophil-related conditions and diseases described herein with respect to treatment.

In some aspects, one or more medical images can be produced within 24 hours after the initiation of the administration of the radiolabeled contrast agent. In some aspects, a first medical image can be produced within 24 hours after the initiation of the administration (or ingestion) of the radiolabeled contrast agent. In some aspects, a first medical image can be produced at any time during the administration (or ingestion) of the radiolabeled contrast agent. In some aspects, any of the methods disclosed herein can further comprise performing a low-dose planar X-ray. In some aspects, the low-dose planar X-ray can be performed 2 hours, 4 hours, 6 hours, 8 hours and/or 24 hours after oral administration of the radiolabeled contrast agent.

Also disclosed herein are methods of diagnosing eosinophilic esophagitis in a subject. In some aspects, the methods can comprise detecting an eosinophil granule protein in the mucosal tissue of the esophagus in a subject. In some aspects, the method can comprise administering to a subject radiolabeled heparin (e.g., radiolabeled high molecular weight heparin) under conditions wherein the radiolabeled heparin can bind to an eosinophil granule protein. In some aspects, the method can comprise detecting a radiolabeled heparin/eosinophil granule protein complex in the mucosal tissue of the esophagus, whereby detecting the radiolabeled heparin/eosinophil granule protein complex in the mucosal tissue of the esophagus diagnoses eosinophilic esophagitis in the subject. In some aspects, the method can comprise administering to a subject radiolabeled heparin under conditions wherein the radiolabeled heparin binds to an eosinophil granule protein to form a radiolabeled heparin/eosinophil granule protein complex. In some aspects, the methods can comprise detecting the radiolabeled heparin/eosinophil granule protein complex in the mucosal tissue of the esophagus. In some aspects, detecting the radiolabeled heparin/eosinophil granule protein complex in the mucosal tissue of the esophagus can diagnose eosinophilic esophagitis in the subject. In some aspects, a radiolabeled heparin/eosinophil granule protein complex can be ^{99m}Tc-heparin/MBP-1.

Further disclosed herein are methods of diagnosing an eosinophilic disease or eosinophil-related inflammation in a subject. In some aspects, the methods comprise administering a composition comprising an effective amount of radiolabeled high molecular weight heparin or a salt thereof and a pharmaceutically acceptable excipient to the subject, wherein the high molecular weight heparin binds to one or more eosinophil granule proteins in the tissue. The methods further comprise detecting the radiolabeled high molecular weight heparin, wherein detecting the radiolabeled high molecular weight heparin in the tissue diagnoses the eosinophilic disease or eosinophil-related inflammation in the subject. In some aspects, detecting the radiolabeled high molecular weight heparin comprises detecting a complex from the radiolabeled heparin binding to the eosinophil granule proteins. The high molecular weight heparin can have a high purity, i.e., a substantial fraction of the heparin chains have a high molecular weight. In some aspects, detecting the radiolabeled high molecular weight heparin comprises detecting a furrow, a ring, and/or a stricture along an esophagus (i.e., a symptom of EoE).

Further disclosed herein are methods of detecting a change in eosinophilic esophagitis in a subject diagnosed with eosinophilic esophagitis. In some aspects, the methods can comprise: (a) producing a first medical image of the esophagus in a subject diagnosed with eosinophilic esophagitis according to the disclosed methods, (b) producing a second medical image of the esophagus in the subject of step (a) according to the disclosed methods, and (c) comparing the medical image of step (b) with the medical image of step (a), whereby detecting a change in the medical image of step (b) compared to the medical image of step (a) detects a change in eosinophilic esophagitis in the subject. In some aspects, the medical image can be three-dimensional. In some aspects, the medical image can be two-dimensional.

Also disclosed herein are methods of monitoring a tissue exhibiting eosinophil-related inflammation in a subject. In some aspects, the methods comprise administering a composition comprising an effective amount of radiolabeled high molecular weight heparin or a salt thereof and a pharmaceutically acceptable excipient to the subject, wherein the high molecular weight heparin binds to one or more eosinophil granule proteins in the tissue. The methods further comprise detecting the radiolabeled high molecular weight heparin to produce a first medical image of the tissue and detecting the radiolabeled high molecular weight heparin to produce a second medical image of the tissue. In some aspects, detecting the radiolabeled high molecular weight heparin comprises detecting a complex from the radiolabeled heparin binding to the eosinophil granule proteins. The methods further comprise comparing the second medical image to the first medical image, whereby detecting a change between the second image and the first image detects a change in the eosinophil-related inflammation of the tissue. The high molecular weight heparin can have a high purity, i.e., a substantial fraction of the heparin chains have a high molecular weight. In some aspects, the heparin is labeled with ^{99m}Tc or another radiolabeled contrast agent or tracer as described herein. In some aspects, the first image and the second image comprise a furrow, a ring, and/or a stricture along an esophagus (i.e., a symptom of EoE).

In some aspects, a first medical image of the esophagus can be produced in a subject diagnosed with EoE to serve as a baseline for future or subsequent comparison with later-produced medical images of the esophagus in the subject. In some aspects, the two or medical images taken at two different time points can be used to determine the change or progression of EoE. In some aspects, a first medical image can be used to determine whether a treatment of EoE is effective (or not effective) in the subject. For example, if a second medical image is produced after the initiation of treatment of EoE in a subject and the second medical image shows fewer areas of radiolabeled heparin/eosinophil granule protein complexes (i.e., inflammation) when compared to the first medical image produced before initiation of treatment, it can indicate that the treatment of EoE in the subject is effective. Conversely, if a second medical image is produced after the initiation of treatment of EoE in a subject and the second medical image shows the same or more areas of radiolabeled heparin/eosinophil granule protein complexes (i.e., inflammation) when compared to the first medical image produced before initiation of treatment, it can indicate that the treatment of EoE in the subject is not effective.

In some aspects, disclosed herein are methods of detecting eosinophil degranulation in a subject. In some aspects, the methods can comprise detecting an eosinophil granule protein in a subject. In some aspects, the method can comprise administering to a subject radiolabeled contrast agent under conditions wherein the radiolabeled contrast agent can bind to an eosinophil granule protein. In some aspects, the methods can comprise detecting a radiolabeled contrast agent/eosinophil granule protein complex, whereby detecting the radiolabeled contrast agent/eosinophil granule protein complex detects eosinophil degranulation in the subject. In some aspects the methods can comprise administering to a subject radiolabeled heparin under conditions wherein the radiolabeled heparin binds to an eosinophil granule protein to form a radiolabeled heparin/eosinophil granule protein complex. In some aspects, the methods can comprise detecting the radiolabeled heparin/eosinophil granule protein complex. In some aspects, detecting the radiolabeled heparin/eosinophil granule protein complex can detect eosinophil degranulation in the subject.

In any of the methods disclosed herein, the organ can be an ovary, a breast, a brain, a muscle, a heart, a lung, a stomach, a proximal large intestine, a distal large intestine, a small intestine, a pancreas, a thyroid, skin, an eye, a testicle, a thymus, a gallbladder, a uterus, an esophagus or a major blood organ. In some aspects, the major blood organ can be the liver, spleen, kidneys, or bladder.

In any of the methods disclosed herein, the eosinophil granule protein can be major basic protein 1 (MBP-1), major basic protein 2 (MBP-2), eosinophil derived neurotoxin (EDN), eosinophil cationic protein (ECP), or eosinophil peroxidase (EPO). In some aspects, the eosinophil granule protein can be MBP-1.

In any of the methods disclosed herein, the radiolabel can be ^{99m}Tc.

Additional tracers, such as tracers used for positron emission tomography, can also be employed for detecting the binding of the HMWH to sites of eosinophilic inflammation. In some aspects, the tracers can be any tracer or label in Table 1.

In any of the methods disclosed herein, the radiolabeled heparin can be administered to the subject orally. In any of the methods disclosed herein, the subject can swallow the radiolabeled heparin through one or more swallows. In any of the methods disclosed herein, the radiolabeled heparin can be administered orally to the subject in 1 ml aliquots over 15 minutes.

In any of the methods disclosed herein, the methods can further comprise a washing step. In some aspects, the washing step can comprise the subject swallowing a liquid after the one or more swallows of the radiolabeled heparin. In some aspects, the liquid can be water. In some aspects, the washing step can be performed before, during or after the production of a medical image. In some aspects, the administration of the liquid can comprise the subject swallowing the liquid through one or more swallows. In some aspects, the liquid can be administered in a volume of 1 to 100 ml.

In any of the methods disclosed herein, the heparin or the heparin portion of the radiolabeled heparin can be high molecular weight heparin, low molecular weight heparin or unfractionated heparin. In some aspects, the heparin or the heparin portion of the radiolabeled heparin can be high molecular weight heparin. In some aspects, the high molecular weight heparin can be administered in an amount less than 1 mg. In some aspects, the high molecular weight heparin can be administered in an amount ranging from 0.1 mg to 1 mg. In some aspects, wherein the radiolabel of the radiolabeled heparin can be administered in an amount ranging from 0.3 mCi to 3 mCi.

In some aspects, the HMWH comprises an average molecular weight of about 20 kDa or greater. For example, the HMWH can comprise an average molecular weight of 20 kDa, 21 kDa, 22 kDa, 23 kDa, 24 kDa, 25 kDa, 26 kDa, 27 kDa, 28 kDa, 29 kDa, 30 kDa, or individual values or ranges therebetween. In some aspects, the HMWH can have an average molecular weight above 30 kDa. In some aspects, the HMWH comprises an average molecular weight of about 35 kDa. In some aspects, the HMWH comprises an average molecular weight of about 40 kDa. It some aspects, the HMWH comprises an average molecular weight greater than 40 kDa. In some aspects, the average molecular weight of the HMWH is an individual value between the values disclosed herein or a range between values disclosed herein.

In some aspects, the predetermined threshold for molecular weight that is used to define the “purity” of the HMWH can be a value other than 20 kDa. The predetermined threshold can be set based on the minimum desired average molecular weight for the HMWH composition. For example, the predetermined threshold for assessing purity of the HMWH can be 20 kDa, 21 kDa, 22 kDa, 23 kDa, 24 kDa, 25 kDa, 26 kDa, 27 kDa, 28 kDa, 29 kDa, 30 kDa, 35 kDa, 40 kDa, greater than 40 kDa, or individual values or ranges therebetween. Similarly, the cutoff of the low molecular weight chains can be a value other than 8 kDa. For example, the cutoff may be 5 kDa, 6 kDa, 7 kDa, 8 kDa, 9 kDa, 10 kDa, 11 kDa, 12 kDa, greater than 12 kDa, or individual values or ranges therebetween.

In any of the methods disclosed herein, the subject can be a human.

Methods of Treating

Currently, heparin has not been used for the treatment of eosinophil-associated diseases, or eosinophil-related inflammation. As disclosed herein, heparin can neutralize the toxicity of eMBP-1. Therefore, the greater avidity of high molecular weight heparin for eMBP-1 can lead to a more effective molecule for neutralizing an eosinophil protein, for example, eMBP-1 and, thus, a more effective treatment for eosinophilic-associated diseases and eosinophil-related inflammation.

Disclosed herein are methods of treating a tissue exhibiting eosinophil-related inflammation in a subject. Also disclosed herein are methods of reducing eosinophil-related inflammation in a tissue. In some aspects, the methods comprise administering a composition comprising an effective amount of high molecular weight heparin or a salt thereof and a pharmaceutically acceptable excipient to the subject. In some aspects, the high molecular weight heparin or salt thereof binds to one or more eosinophil granule proteins in the tissue. The high molecular weight heparin or salt thereof may have a high purity, i.e., a substantial fraction of the heparin chains have a high molecular weight. In some aspects, the methods comprise administering a composition comprising an effective amount of unfractionated heparin or a salt thereof and a pharmaceutically acceptable excipient to the subject. In some aspects, the unfractionated heparin or salt thereof binds to one or more eosinophil granule proteins in the tissue.

Disclosed herein are methods of treating eosinophilic-related inflammation in a subject. The methods can comprise: administering a therapeutically effective amount of a composition comprising an effective amount of heparin having an average molecular weight from about 20 kDa to about 40 kDa. In some aspects, at least 50% of heparin chains in the heparin have a molecular weight of at least 20 kDa. In some aspects, the method can comprise administering a therapeutically effective amount of a composition comprising an effective amount of unfractionated heparin. In some aspects, the composition further comprise a pharmaceutically acceptable excipient. In some aspects, the heparin comprises an average molecular weight of at least 20 kDa. In some aspects, the heparin comprises an average molecular weight of at least 30 kDa. In some aspects, the heparin comprises an average molecular weight of at least 40 kDa. In some aspects, at least 60% of heparin chains in the heparin have a molecular weight of at least 20 kDa. In some aspects, at least 70% of heparin chains in the heparin have a molecular weight of at least 20 kDa. In some aspects, the therapeutically effective dose of heparin is about 3 mg. In some aspects, the therapeutically effective dose of heparin is about 1 mg. In some aspects, the therapeutically effective dose of heparin is about 0.5 mg. In some aspects, the heparin is configured to bind to one or more eosinophil granule proteins. In some aspects, the binding affinity of the heparin for the one or more eosinophil granule proteins is greater than the binding affinity of a low molecular weight heparin for the one or more eosinophil granule proteins.

Disclosed herein are methods of delivering a therapeutic agent to a diseased tissue, organ, or body part. In some aspects, the methods include delivering a therapeutic agent to a diseased organ. Disclosed herein are methods of treating one or more eosinophilic diseases or eosinophilic-associated diseases in a subject. In some aspects, the methods can comprise administering a therapeutically effective amount of a composition comprising heparin conjugated to a therapeutic agent to a subject. In some aspects, the heparin can be high molecular weight heparin. In some aspects, the heparin can be unfractionated heparin.

In some aspects, the compositions disclosed herein can further comprise a therapeutic agent conjugated to the HMWH. In some aspects, the compositions disclosed herein can further comprise a therapeutic agent conjugated to the unfractionated heparin. In some aspects, the compositions can further comprise a therapeutically effective amount of the therapeutic agent for administration to the patient. In some aspects, the therapeutic agent is configured to have a therapeutic effect on the eosinophil-related inflammation and/or disease. Accordingly, in some aspects, a therapeutic effect of the high molecular weight heparin and a therapeutic effect of the therapeutic agent can be used in combination on a site of eosinophil-related inflammation and/or disease. As disclosed herein, by conjugating therapeutic agents to HMWH, a treatment can be targeted directly to an area(s) of inflammation because the avidity of the HMWH for tissue bound eMBP-1. Thus, the targeting of the HMWH/therapeutic agent complex directly to one or more sites of eosinophil-related inflammation can reduce the quantity (or dose) of the therapeutic agent needed for care, and thus limit any side effects associated with the administration of the therapeutic agent. Accordingly, the therapeutically effective amount of the therapeutic agent can be less than a therapeutically effective amount typically associated with administration of the therapeutic agent in the absence of HMWH or another targeted mechanism. In some aspects, a therapeutic effect of the unfractionated heparin and a therapeutic effect of the therapeutic agent can be used in combination on a site of eosinophil-related inflammation and/or disease. As disclosed herein, by conjugating therapeutic agents to unfractionated heparin, a treatment can be targeted directly to an area(s) of inflammation because the avidity of the HMWH for tissue bound eMBP-1. Accordingly, the therapeutically effective amount of the therapeutic agent can be less than a therapeutically effective amount typically associated with administration of the therapeutic agent in the absence of unfractionated heparin or another targeted mechanism. In some aspects, the therapeutic agent is a glucocorticoid, which is an effective treatment for eosinophil-related diseases. In some aspects, the glucocorticoid is one or more of mometasone, fluticasone, budesonide, and methylprednisolone. Additional therapeutic agents for eosinophil-related inflammation or diseases are contemplated as would be apparent to one having an ordinary level of skill in the art.

Disclosed herein are methods of treating eosinophilic-related inflammation in a subject. In some aspects, the methods can comprise administering a therapeutically effective amount of a composition comprising: heparin conjugated to a therapeutic agent to a subject. In some aspects, the heparin can be high molecular weight heparin. In some aspects, the heparin can be unfractionated heparin. In some aspects, the composition can be administered to the subject orally, intravenously, by inhalation, optically or topically.

In some aspects, the HMWH comprises an average molecular weight of about 20 kDa or greater. For example, the HMWH can comprise an average molecular weight of 20 kDa, 21 kDa, 22 kDa, 23 kDa, 24 kDa, 25 kDa, 26 kDa, 27 kDa, 28 kDa, 29 kDa, 30 kDa, or individual values or ranges therebetween. In some aspects, the HMWH can have an average molecular weight above 30 kDa. In some aspects, the HMWH comprises an average molecular weight of about 35 kDa. In some aspects, the HMWH comprises an average molecular weight of about 40 kDa. It some aspects, the HMWH comprises an average molecular weight greater than 40 kDa. In some aspects, the average molecular weight of the HMWH is an individual value between the values disclosed herein or a range between values disclosed herein.

The average molecular weight of the HMWH can be selected to optimize binding to sites expressing eosinophilic inflammation. Because HMWH exhibits a higher affinity for MBP-1 than low molecular weight heparin (LMWH) or unfractionated heparin (UFH), HMWH will bind more avidly than LMWH or unfractionated heparin UFH to sites of eosinophilic inflammation. In some aspects, a HMWH with a relatively high average molecular weight (e.g., 30 kDa) can bind more avidly than a HMWH with a relatively low average molecular weight (e.g., 20 kDa). In some aspects, the binding affinity of the HMWH increases linearly with the average molecular weight of the HMWH. Accordingly, as the average molecular weight of the HMWH increases, the quantity of heparin required for localization of eosinophilic inflammation can be reduced with the expectation that a greater percentage of administered heparin will localize to the inflammation sites and will neutralize toxic cationic eosinophil proteins.

The purity of the HMWH may be defined by the amount of heparin chains having a molecular weight above a predetermined threshold. For example, the predetermined threshold may be 20 kDa and accordingly the purity of the HMWH can be determined based on a fraction, percentage, or ratio of heparin chains having a molecular weight of 20 kDa or greater compared to those having a molecular weight of less than 20 kDa. In some aspects, at least about 50% of the heparin chains in the HMWH may have a molecular weight of 20 kDa or greater, which may also be referred to as a purity of 50% (i.e., “high purity”). In some aspects, the total percentage of heparin chains in the HMWH having a molecular weight of 20 kDa or greater may be 60%, 70%, 80%, 90%, 95%, greater than 95%, or individual values or ranges therebetween. Accordingly, the composition of HMWH can be described as having 60% purity, 70% purity, 80% purity, 90% purity, 95% purity, greater than 95% purity, or individual values or ranges therebetween. In some aspects, the HMWH can also be defined by a maximum amount of molecular chains with a molecular weight below the predetermined threshold. For example, the HMWH can comprise a percentage of heparin chains with a molecular weight below 20 kDa at or below 50%, 40%, 30%, 25%, 20%, 15%, 10%, 5%, less than 5%, or individual values or ranges therebetween. In some aspects, the HMWH can be additionally defined by a maximum amount of molecular chains having a molecular weight below a cutoff defining low molecular weight chains (e.g., 8 kDa). For example, the HMWH can comprise a percentage of heparin chains with a molecular weight below 8 kDa at or below 50%, 40%, 30%, 25%, 20%, 15%, 10%, 5%, less than 5%, or individual values or ranges therebetween.

In some aspects, a HMWH with a relatively high purity (e.g., 80%) can demonstrate greater localization to the eosinophil-related inflammation site than a HMWH with a lower purity (e.g., 50%). In some aspects, the localization rate of the HMWH increases as the purity of the HMWH increases. Accordingly, as the purity of the HMWH increases, the quantity of heparin required for adequate localization of eosinophilic inflammation can be reduced with the expectation that a greater percentage of administered heparin will localize to the inflammation sites.

Additionally, in some aspects the predetermined threshold for molecular weight that is used to define “purity” of the HMWH can be a value other than 20 kDa. The predetermined threshold can be set based on the minimum desired average molecular weight for the HMWH composition. For example, the predetermined threshold for assessing purity of the HMWH may be 20 kDa, 21 kDa, 22 kDa, 23 kDa, 24 kDa, 25 kDa, 26 kDa, 27 kDa, 28 kDa, 29 kDa, 30 kDa, 35 kDa, 40 kDa, greater than 40 kDa, or individual values or ranges therebetween. Similarly, the cutoff of the low molecular weight chains can be a value other than 8 kDa. For example, the cutoff can be 5 kDa, 6 kDa, 7 kDa, 8 kDa, 9 kDa, 10 kDa, 11 kDa, 12 kDa, greater than 12 kDa, or individual values or ranges therebetween.

In a case where high purity is defined by a relatively higher threshold (e.g., 30 kDa), the HMWH can demonstrate greater localization to the eosinophil-related inflammation site than a case where high purity is defined by a relatively lower threshold (e.g., 20 kDa). In some aspects, the localization rate of the HMWH increases as the purity threshold increases. Accordingly, as the purity threshold of the HMWH increases, the quantity of heparin required for adequate localization of eosinophilic inflammation can be reduced with the expectation that a greater percentage of administered heparin will localize to the inflammation sites.

In a case where high purity is defined by a relatively higher threshold (e.g., 30 kDa), the HMWH can demonstrate greater localization to the eosinophil-related inflammation site than a case where high purity is defined by a relatively lower threshold (e.g., 20 kDa). In some aspects, the localization rate of the HMWH increases as the purity threshold increases. Accordingly, as the purity threshold of the HMWH increases, the quantity of heparin required for treatment of or reduction in eosinophilic inflammation can be reduced with the expectation that a greater percentage of administered heparin will localize to the inflammation sites.

The compositions described herein can comprise a specified quantity of HMWH heparin. In some aspects, the specified quantity of HMWH can be a dose of HMWH configured to treat (or to reach) an eosinophil-related inflammation site. In some aspects, the specified quantity of HMWH can be a therapeutically effective amount of HMWH. In some aspects, the specified quantity of HMWH can be a dose of HMWH configured to localize to the eosinophil-related inflammation site and facilitate imaging and/or diagnosis thereof. For example, where the eosinophil-related inflammation site is an esophagus, the composition can comprise a quantity of HMWH selected from about 15000 units, about 10000 units, about 5000 units, about 4000 units, about 3000 units, about 2000 units, about 1000 units, about 500 units, about 250 units, less than about 250 units, or individual values or ranges therebetween. The quantity of HMWH may be about 100 mg, about 90 mg, about 80 mg, about 70 mg, about 60 mg, about 50 mg, about 40 mg, about 30 mg, about 20 mg, about 10 mg, about 5 mg, about 4 mg, about 3 mg, about 2 mg, about 1 mg, about 0.5 mg, less than about 0.5 mg, or individual values or ranges therebetween. In some embodiments, the quantity of heparin may be diluted (e.g., with sterile saline) to provide a final volume of about 10 mL, about 9 mL, about 8 mL, about 7 mL, about 6 mL, about 5 mL, about 4 mL, about 3 mL, about 2 mL, about 1 mL, about 0.9 mL, about 0.8 mL, about 0.7 mL, about 0.6 mL, about 0.5 mL, about 0.4 mL, about 0.3 mL, about 0.2 mL, about 0.1 mL, less than about 0.1 mL, or individual values or ranges therebetween. The dose of HMWH can vary based on the size of the targeted eosinophil-related inflammation site. A larger quantity of HMWH may be required for targeting larger sites and/or organs. Where the eosinophil-related inflammation site is a different site or organ other than the esophagus as further described herein, the quantity of HMWH can be a value described herein or a larger or small value necessary to adequately target the eosinophil-related inflammation site as would be apparent to one having an ordinary level of skill in the art.

In some aspects, the compositions can comprise unfractionated heparin. In some aspects, the unfractionated heparin can be heparin sodium. In some aspects, the heparin sodium can be 1000 USP units, 5000 USP units, 10,000 UPS units or any amount in between.

As described herein, the compositions generally comprise a relatively small quantity of HMWH heparin because the high affinity for MBP-1 and high purity of the composition results in a lower required dose as compared to LMWH or UFH. Accordingly, the small quantity of HMWH poses a relatively low risk of HIT because the total quantity of heparin administered is low compared to commonly acceptable doses of LMWH or UFH. Further, even LMWH and UFH commonly include a quantity of high molecular weight chains due to their low purity (i.e., high polydispersity). Accordingly, in some cases the total quantity of high molecular weight chains in the composition can be substantially similar to the total quantity of high molecular weight chains found in typically acceptable doses of LMWH or UFH, and thus do not pose a substantially greater risk of HIT. Further, when HMWH is administered orally, by inhalation, or topically as described herein, the risk of HIT may be greatly diminished in comparison to the degree of risk typically associated with administration of heparin intravenously and/or subcutaneously because both oral and topical administration of HMWH is not absorbed and thus does not cause anticoagulation.

In some aspects, the compositions disclosed herein can be administered orally or topically as an oral or topical solution. For example, compositions comprising UFH or HMWH can be formulated as an oral solution or a topical solution for treating eosinophilic GI disorders (EGIDs), including by not limited to EoE and eosinophilic gastroenteritis; and inflammatory bowel disease, including by not limited to ulcerative colitis and Crohn's disease.

In some aspects, the compositions disclosed herein can be administered by inhalation as a nasal spray. For example, compositions comprising UFH or HMWH can be formulated as a nasal spray for treating eosinophilic chronic rhinosinusitis or nasal polyps.

In some aspects, the compositions disclosed herein can be administered topically (e.g., eye drops). For example, compositions comprising UFH or HMWH can be formulated for topical administration for treating ocular diseases having an allergic pathophysiological component including but not limited to eosinophilic conjunctivitis, seasonal and/or perennial allergic conjunctivitis, vernal conjunctivitis, atopic keratoconjunctivitis, giant papillary conjunctivitis or contact dermatoconjunctivitis.

In some aspects, the compositions can be administered orally. For example, the composition can be swallowed orally by the subject. In another example, the composition can be administered orally with a syringe, dropper, or other device. In some aspects, the composition can be administered over a period of time. In some aspects, the composition is administered over 5 minutes. However, the composition may be administered over about 1 minute, about 2 minutes, about 3 minutes, about 4 minutes, about 6, minutes, about 7 minutes, about 8 minutes, about 9 minutes, about 10 minutes, greater than about 10 minutes, or individual values or ranges therebetween. In some aspects, the composition is administered over the period of time in discrete portions or aliquots. For example, the composition can be administered or swallowed orally by the subject over about 5 minutes in about 1 ml aliquots (e.g., about 1 ml/minute). In some aspects, the subject can perform 5 swallows of about 1 ml of the composition. However, the number of swallows of the composition can be 1, 2, 3, 4, 6, 7, 8, 9, 10, 11, 12, 13, 14, 15, greater than 15, or ranges therebetween. In some embodiments, the aliquots or swallows may comprise about 1 mL, about 2 mL, about 3 mL, about 4 mL, about 5 mL, more than about 5 mL, or individual values or ranges therebetween. In some aspects, the composition is administered to a subject while the subject is in the supine position. In some aspects, the subject remains in the supine position for about 1 minute, about 5 minutes, about 10 minutes, about 15 minutes, about 20 minutes, about 25 minutes, about 30 minutes, or individual values or ranges therebetween. In some aspects, the subject does not eat or drink for a specified period of time after administration. In some aspects, the subject does not eat or drink for about 1 minute, about 5 minutes, about 10 minutes, about 15 minutes, about 20 minutes, about 25 minutes, about 30 minutes, or individual values or ranges therebetween. In some embodiments, the subject swallows water after administration or after remaining in the supine position for a period of time. In some aspects, the subject swallows about 100 ml of water after remaining in the supine position for at least about 15 minutes. However, the subject can swallow water in the amount of about 1 mL, about 5 mL, about 10 mL, about 15 mL, about 20 mL, about 25 mL, about 30 mL, about 35 mL, about 40 mL, about 45 mL, about 50 mL, about 55 mL, about 60 mL, about 65 mL, about 70 mL, about 75 mL, about 80 mL, about 85 mL, about 90 mL, about 95 mL, about 100 mL, greater than about 100 mL, or individual values or ranges therebetween. In some aspects, the subject can perform 15 swallows of about 6-7 ml of the composition. However, the number of swallows of the composition can be 1, 2, 3, 4, 6, 7, 8, 9, 10, 11, 12, 13, 14, 15, greater than 15, or ranges therebetween. In some aspects, the swallows may comprise about 1 mL, about 2 mL, about 3 mL, about 4 mL, about 5 mL, about 6 mL, about 7 mL, about 8 mL, about 9 mL, about 10 mL, greater than about 10 mL, or individual values or ranges therebetween. In some aspects, the patient swallows water after each swallow or aliquot of the composition. In some aspects, the patient waits a specified period of time as described above between each swallow or aliquot of the composition. In some aspects, when the compositions are administered for the purpose of treating eosinophilic inflammation, the subject does not need to swallow water after administration. In some aspects, heparain (e.g., HMWH) can be administered orally without being conjugated to a radiolabel for treating or reducing eosinophilic inflammation, and said administered does not include a step of swallowing any amount of water after administration. Additional manners or procedures for administration are contemplated as would be apparent to one having an ordinary level of skill in the art.

In some aspects, the composition is administered by another route. For example, where the composition is administered to treat other eosinophil-related conditions and diseases than eosinophilic esophagitis, different administration routes may be necessary or preferable. In some embodiments, the compositions disclosed herein are configured for administration intravenously, topically, by inhalation, and/or orally to treat gastrointestinal eosinophil-associated diseases. In some embodiments, the gastrointestinal eosinophil-associated diseases that can be treated by oral (or topical) administration comprise eosinophilic esophagitis, eosinophilic gastritis, and/or eosinophilic gastroenteritis. In some embodiments, the compositions disclosed herein are configured for administration by inhalation to treat inflammation in the nose, paranasal sinuses and lung. In some aspects, the compositions disclosed herein are configured for administration by an enema to treat the colon. In some embodiments, the compositions disclosed herein are configured for administration by catheter to treat eosinophil-related inflammation in the urinary bladder. In some aspects, the compositions disclosed herein are configured for administration by eye drops to treat ocular eosinophilic-related inflammation or diseases having an allergic pathophysiological component. In some aspects, the compositions disclosed herein are configured for topical administration as a cream or ointment to treat eosinophil-related inflammation and/or diseases of the skin.

While the composition is substantially described in regards to administration to an esophagus, the composition can be configured (or formulated) for administration to additional tissues or organs. In some aspects the targeted eosinophil-related inflammation or eosinophilic disease may be specific to the gastrointestinal tract (e.g., mouth, esophagus, stomach, small intestine, large intestine, or colon) lung, nose, eye, skin, one or more joints, one or more muscles, one or more nerves, heart, kidney, bladder, uterus, prostate, breast, lymph or blood.

In some aspects, the eosinophil-related inflammation or eosinophilic disease can be any of the diseases or disorders or syndromes disclosed herein. Additional eosinophil-related inflammation and eosinophilic diseases are contemplated here as would be apparent to one having an ordinary level of skill in the art.

In some aspects, the therapeutic agent can be a glucocorticoid. In some aspects, the glucocorticoid can be mometasone, fluticasone, budesonide, prednisone or solumedrol. In some aspects, heparin can be conjugated with one or more glucocorticoids. Glucocorticoids are effective treatments for eosinophil-related diseases. As disclosed herein, by conjugating glucocorticoids to heparin, a treatment can be targeted directly to an area(s) of inflammation because the avidity of the heparin-glucocorticoid complex (e.g., heparin conjugated to a glucocorticoid) for tissue bound eMBP-1. Further, more efficient (and selective) targeting of the heparin-glucocorticoid complex directly to one or more sites of eosinophil-related inflammation may reduce the quantity (or dose) of the glucocorticoid needed for care, and, thus, limit any side effects associated with the administration of glucocorticoids.

In some aspects, the diseased tissue, organ or body part can be any tissue, organ or body part disclosed herein. In some aspects, the diseased tissue or organ or body part can be subcutaneous fat, fascia, muscle, endomysium, fibrous tissue, mesentery, an ovary, a breast, a brain, a muscle, a heart, a lung, a stomach, a proximal large intestine, a distal large intestine, a small intestine, a pancreas, a thyroid, skin, mucous membrane, an eye, a testicle, a thymus, a gallbladder, a uterus, a liver, a spleen, a kidney, an esophagus, a bladder, a bile ducts, a blood vessel, a sinus, a larynx, a trachea, a thymus, a nerve, spinal cord, ganglia, diaphragm or a major blood organ. In some aspects, the diseased tissue, organ or body part can be nasal and/or sinus mucosa. In some aspects, the major blood organ can be liver, spleen, kidneys, or bladder.

In some aspects, in any of the methods disclosed herein, disclosed herein is a step comprising identifying the subject in need thereof. In some aspects, the subject in need thereof can be identified by any of the methods disclosed herein. In some aspects, the subject can be a human.

Methods of Making the Compositions Described Herein

Disclosed herein are methods of making high molecular weight heparin compositions. In some aspects, the method comprises using gel permeation chromatography to measure molecular weight, and purify and isolate HMWH heparin. In some aspects, the methods of making high molecular weight heparin compositions can be carried out using any method known to one of ordinary skill in the art for separating molecules of differing molecular weights.

In some aspects, the method can comprise lyophilizing heparin, resuspending the lyophilized heparin in water and applying the resuspended heparin to a chromatography column. By using lyophilized heparin, the (resuspended) heparin applied to the chromatography column can be concentrations so that a greater quantity of the heparin can be applied to the column. In some aspects, the heparin is not lyophilized and resuspended before being applied to the chromatography column. In some aspects, the heparin can be preservative free heparin or medicinal grade heparin. The concentration of heparin used in the methods disclosed herein can depend on the capacity of the column selected. In some aspects, the column can be a gel permeation column. In some aspects, the column can be a polyacrylamide gel (e.g., Bio-Gel P-60) column. In some aspects, heparin can be fractionated on a column of polyacrylamide gel (e.g., Bio-Gel P-60). In some aspects, any column can be used. In some aspects, any column known to one of ordinary skill in the art can be used for separating molecules of differing molecular weights. In some aspects, the heparin can be preservative free heparin or medicinal grade heparin. In some aspects, medicinal grade heparin, USP, 60 ml, 2000 units per ml, can be lyophilized (weight of powder 0.9 gm), and the powder can be resuspended in water, and about 2.0 mL to about 3.0 mL (e.g., 2.4 mL) of the resuspended heparin can be applied to the column. In some aspects, heparin can detected by absorbance at about 232 nm. In some aspects, blue dextran (molecular mass of about 2,000 kDa) and vitamin B12 (molecular mass of 1356 Da) can be used to calibrate the column. In some aspects, blue dextran can elute at about 33 mL and vitamin B12 can elute at about 100 mL. In some aspects, heparin can be contained in fractions eluting between about 33 mL and 80 mL. In some aspects, high molecular weight heparins were contained in the fractions eluting from about 33 mL to about 50 mL.

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples:

### EXAMPLES

### Example 1 Oral Administration of ^{99m}Technetium-Labelled Heparin in Patients with Eosinophilic Esophagitis

Eosinophilic esophagitis (EoE) is an inflammatory disease of the esophagus that constitutes the second most prevalent cause of chronic esophagitis, the first being gastroesophageal reflux disease (GERD) (Hiremath G, et al. Dig Dis Sci 2018; and Hiremath G S, et al. Dig Dis Sci 2015; 60:3181-93). Considerable evidence reveals that EoE, which commonly affects children, adolescents and young adults, is increasing in prevalence (Noel R J, et al. N Engl J Med 2004; 351:940-1; Prasad G A, et al. Clin Gastroenterol Hepatol 2009; 7:1055-61; and Dellon E S, et al. Clin Gastroenterol Hepatol 2014; 12:589-96 el). The disease frequently presents with difficulty swallowing (dysphagia) and, occasionally, food becomes lodged in the esophagus obstructing swallowing and leading to emergency hospital visits (Liacouras C A, et al. J Allergy Clin Immunol 2011; 128:3-20 e6; quiz 21-2). The onset can be insidious, and, for that reason and others, the diagnosis often is delayed with the potential development of irreversible sequelae (Schoepfer A M, et al. Gastroenterology 2013; 145:1230-6 el-2).

The diagnosis of EoE is made based on characteristic features in the patient's medical history and findings from esophagogastroduodenoscopy (EGD) with multiple esophageal biopsies (Furuta G T, Katzka D A. N Engl J Med 2015; 373:1640-8). Because the disease affects the esophagus in an irregular, “spotty” manner, at least five, and, preferably a greater number of, biopsies should be obtained (Gonsalves N, Kahrilas P J. Neurogastroenterol Motil 2009; 21:1017-26). Tissue specimens showing 15 or more eosinophils per high power field (HPF) satisfy a diagnostic criterion for EoE. Evaluation and management of the disease in current practice is challenging: first, there is the necessity of performing endoscopy with conscious (moderate) sedation each time the disease is appraised; second, conscious (moderate) sedation disables the patient for the day; third, the results of histopathological examination with eosinophil counts on the biopsies are not available for several days to over a week; fourth, because the eosinophilic inflammation in the esophagus is irregular, “spotty,” even performance of multiple biopsies may not yield results that reflect disease activity accurately or completely, including symptoms that patients experience; and, last, endoscopy and biopsy evaluations are expensive (Saffari H, et al. J Allergy Clin Immunol 2012; 130:798-800; and Salek J, et al. Aliment Pharmacol Ther 2015; 41:1288-95).

Previous studies have shown that eosinophils release their distinctive, markedly cationic granule proteins, including eosinophil major basic protein-1 (eMBP-1), into the tissues of the affected esophagus. This deposition may be a better indicator of the disease activity than eosinophil counts (Kephart G M, et al. Am J Gastroenterol 2010; 105:298-307; Peterson K A, et al. Dig Dis Sci 2015; 60:2646-53; and Saffari H, et al. Am J Gastroenterol 2016; 111:933-9). Examination of esophageal specimens by electron microscopy showed that about 81 percent of eosinophils in the tissues have disrupted membranes, (Saffari H, et al. J Allergy Clin Immunol 2014; 133:1728-34 el) and examination by immunostaining demonstrated the tissue is painted with granule proteins deposited outside of cells (Salek J, et al. Aliment Pharmacol Ther 2015; 41:1288-95; and Kephart G M, et al. Am J Gastroenterol 2010; 105:298-307). (Protheroe et al., Clin Gastroenterol Hepatol 2009, 7:749-755). With the recognition that markedly basic eosinophil granule proteins are deposited on the inflamed esophagus in EoE, it was tested whether inflammation could be localized using ^{99m}Technetium labeled heparin (Tc99m-heparin). Heparin is a markedly acidic molecule normally present in the body that can be labeled for radiological detection, and binds avidly to eMBP-1 forming a crystallographic complex (Swaminathan G J, et al. Biochemistry 2005; 44:14152-8; Swaminathan G J, et al. J Biol Chem 2001; 276:26197-203; Gleich G J, et al. Proc Natl Acad Sci USA 1986; 83:3146-50; and Gleich G J, et al. J Exp Med 1974; 140:313-32). A prior study showed that Tc99m-heparin identifies inflammation in ex vivo biopsy specimens from patients with EoE and not in patients unaffected with EoE, indicating the feasibility of using Tc99m-heparin to image EoE (Saffari H, et al. J Allergy Clin Immunol 2013).

Described herein, the feasibility, biodistribution, and radiation dosimetry of oral administration of Tc99m-heparin in five patients was determined.

Patients. Five patients, including one control without EoE and four patients with EoE, were studied after signing informed consent. Patient 1 was male, 34 years old, with intermittent reflux symptoms consistent with GERD who had not undergone prior EGD. Reflux symptoms were responsive to H2-receptor blocking antihistamine therapy, which he took intermittently. Patient 2 was a 38-year-old male with EoE, longstanding dysphagia and significant pan-esophageal stenosis with a prior peak eosinophil count of 22/HPF and endoscopy scoring of E1R2E0F1S1 (EREFS scoring: E=Edema, R=Rings, E=exudates, F=furrows, S=strictures and the scores range) (Dellon E S, et al. Clin Gastroenterol Hepatol 2016; 14:31-9). Patient 3 was a 53-year-old male recently diagnosed with EoE with severe painful swallowing (odynophagia) treated with 40 mg omeprazole having had a peak eosinophil count of 70/HPF and endoscopy scoring of E1R2E2F1S1. Patient 4 was a 29-year-old male, with EoE and a peak eosinophil count of 40/HPF. After 8 weeks of omeprazole treatment, 40 mg a day, endoscopy was repeated with E1R0E0F1S0 scoring and peak eosinophil count of 35/HPF. He complained of continuing episodic dysphagia, occurring at least 2 times a week. Patient 5 was a 34-year-old male with a history of gastric ulceration (gastrointestinal bleeding) and EoE with dysphagia. His prior endoscopy demonstrated a peak eosinophil count of 45/HPF with endoscopy scoring of E1R0E0F1S0, and he remained symptomatic for over two months on high dose proton pump inhibitor therapy.

Subsequent analyses presented below revealed that patients 1 and 4 had no evidence of active eosinophil-related inflammation at the time of the imaging evaluation; patient 4 had been further treated, and inflammation was not present. In contrast, patients 2, 3 and 5 had evidence of active eosinophil-related inflammation manifested at the time of testing.

Tc99m-Heparin Labeling. On the day of imaging, 20 mg/mL stannous chloride dihydrate (Sigma-Aldrich, MO; product no. 31669) in sterile water was prepared under flowing medical-grade nitrogen, and 0.3 mL solution was filtered through a 0.22 micro filter and mixed with 88 mg (1.5 mL, 5000 units/0.5 mL) of heparin sodium injection (i.e., unfractionated; APP Pharmaceuticals LLC, Barceloneta, PR; NDC 63323-543-02). Tc-99m, 30 mCi was calibrated for the time of patient administration and eluted in 0.4 mL from the Tc-99m generator (Lantheus Medical Imaging, Billerica MA). It was then added to the heparin solution and incubated about 5 minutes at 20° C. Quality control was performed by thin layer chromatography (Whatman no. 31, chromatography strip No. 150-001, Biodex Medical Systems, Shirley, NY) in acetone following the manufacturer's instruction. For oral administration to patients, the radiolabeled solution was diluted in sterile saline, bringing the final volume to 15 mL.

Imaging Protocol. Patients fasted overnight before the study. Fiducial markers, 20 μCi Tc-99m, were placed at 6 sites: sternal notch, both breast nipples, umbilicus, and both iliac crests. Approximately 30 mCi of Tc99m-heparin was administered orally over 15 minutes (1 mL/minute) with the patient swallowing 1 ml aliquots administered with a syringe while lying on his back. During ingestion of the Tc99m-heparin, dynamic imaging (60 frames/minute, 15 minutes, 900 frames) was performed using a dual head single-photon emission computed tomography (SPECT/CT) camera (Symbia Intevo, Siemens Healthineers, Hoffman Estates, IL) over the chest and upper abdomen. The patient then rested, reclined in a flat position, for 15 minutes and, at the 30 minute mark, ingested 100 ml of water followed by “eyes to thighs” SPECT/CT imaging. After another 30 minutes in a reclined, flat position, whole-body anterior and posterior planar images were performed at about the 60 minute mark. Each patient was then permitted to leave the imaging area but returned for five additional anterior and posterior whole-body planar imaging sessions at approximately 2 hours, 4 hours, 6 hours, 8 hours and 24 hours after oral administration. A low-dose planar x-ray (topogram) of the thorax, abdomen, pelvis and proximal lower extremities was performed at each of these time points to assist with Tc99m-heparin localization.

The whole-body, abdominal organs, and esophageal radioactivity counts were recorded from both anterior and posterior planar images at each time point. Regions-of-interest were manually defined on planar images using Siemens software (Symbia.Net Siemens Healthineers, Hoffman Estates, IL), and the geometric means were calculated and recorded. The SPECT/CT and planar images were further analyzed using OsiriX DICOM (Digital Imaging and Communications in Medicine) Viewer software (Pixmeo SARL, Bemex, Switzerland).

Visual Assessment. Three observers graded and recorded visual intensities scores of Tc99m-heparin binding for the proximal, mid and distal esophagus on a visual analog scale of 0 to 4 (0=no uptake, 1=subtle uptake, 2=mild uptake, 3=moderate uptake but less than intensity in bowel, and 4=uptake similar to bowel).

Endoscopy, Histopathology and Immunostaining. Patients underwent EGD one day after completion of the imaging procedure. Biopsy specimens were collected from proximal, mid and distal esophagus (total of about nine per patient) and were submitted for histopathological examination and for eMBP-1 granule protein staining. Peak eosinophils counts per HPF were determined by histopathology on hematoxylin and eosin-stained sections of formalin-fixed specimens, as in current practice, for each individual. For immunostaining, biopsy specimens were transported in Michel's medium, washed and cryo-embedded into one block from each site. The specimen blocks were cryo-sectioned. Serial sections were stained with polyclonal antibody to eMBP-1 by indirect immunofluorescence and examined by fluorescence microscopy to identify intact eosinophils and extracellular eosinophil granule protein deposition. The antibody-stained sections were compared to serial sections stained with normal rabbit IgG (as negative control) and graded on a visual analog scale with reference images from negative (no detectable eMBP-1) to 3+(Talley N J, et al. Gastroenterology 1992; 103:137-45). Additionally, a hematoxylin and eosin-stained section was comparatively examined for morphological features and orientation.

Dosimetry. Anterior and posterior counts of the esophagus at the various time points were converted to percent-injected radioactivity. Values of percent-injected activity per organ over time were fit using the Simulation, Analysis, and. Modeling Software II (SAAM II), software (Epsilon Group, Charlottesville, VA). Time integrals of activity were entered into the Organ Level INternal Dose Assessment/EXponential Modeling (OLINDA/EXM) 2.0®dyna (OLINDA/EXM) 2.0 software, using the adult male model (Stabin M G, Siegel J A. J Nucl Med 2018; 59:154-160). Swallowed Tc99m-heparin was assumed to follow the standard kinetics of the International Commission on Radiological Protection (ICRP) Human Alimentary Tract (HAT) model (ICRP 2006).

Statistical Analysis. Spearman rank correlation coefficients were calculated using IBM SPSS Statistics V25 for the relationships between visual grading scores of Tc99m-heparin binding intensity and peak eosinophil counts/HPF, the eMBP-1 immunostaining grades, and the endoscopy scores of esophageal disease, and relationship between the geometric mean counts of planar images and the eMBP-1 immunostaining grades.

Analyses of the freshly prepared Tc99m-heparin showed that more than 95 percent of the Tc-99m was bound to heparin. Tc99m-heparin administration was well tolerated in the patients and was not appreciably absorbed through the gastrointestinal tract. None of the patients reported any adverse reactions. The majority of radioactivity decayed after 24 hours as confirmed by planar images.

Whole-body biodistribution of Tc99m-heparin was determined in the patients. In addition, more detailed biodistribution and tracer kinetics were measured in esophageal tissue up to about 24 hours post-injection. Table 5 summarizes radiation dose estimates for individual organs and for the total body.

TABLE 2
The human radiation doses estimates based
on adult male model (mGy/MBq administered).

| | Patient 1 | Patient 2 | Patient 3 | Patient 4 | Patient 5 | Average |
|---|---|---|---|---|---|---|
| Adrenals | 1.15E−02 | 1.15E−02 | 1.09E−02 | 1.15E−02 | 1.15E−02 | 1.14E−02 |
| Brain | 2.50E−05 | 2.59E−05 | 3.20E−05 | 2.47E−05 | 2.49E−05 | 2.65E−05 |
| Esophagus | 5.78E−03 | 8.00E−03 | 2.47E−02 | 4.91E−03 | 5.53E−03 | 9.78E−03 |
| Eyes | 2.36E−05 | 2.44E−05 | 2.93E−05 | 2.34E−05 | 2.35E−05 | 2.48E−05 |
| Gallbladder Wall | 2.05E−02 | 2.05E−02 | 1.94E−02 | 2.06E−02 | 2.05E−02 | 2.03E−02 |
| Left colon | 1.15E−01 | 1.15E−01 | 1.09E−01 | 1.16E−01 | 1.15E−01 | 1.14E−01 |
| Small Intestine | 6.59E−02 | 6.57E−02 | 6.22E−02 | 6.61E−02 | 6.58E−02 | 6.51E−02 |
| Stomach Wall | 4.28E−02 | 4.27E−02 | 4.06E−02 | 4.29E−02 | 4.27E−02 | 4.23E−02 |
| Right colon | 1.58E−01 | 1.58E−01 | 1.50E−01 | 1.59E−01 | 1.58E−01 | 1.57E−01 |
| Rectum | 5.31E−02 | 5.29E−02 | 5.02E−02 | 5.33E−02 | 5.31E−02 | 5.25E−02 |
| Heart Wall | 5.33E−03 | 5.33E−03 | 5.23E−03 | 5.33E−03 | 5.32E−03 | 5.31E−03 |
| Kidneys | 8.75E−03 | 8.72E−03 | 8.29E−03 | 8.77E−03 | 8.74E−03 | 8.65E−03 |
| Liver | 6.97E−03 | 6.95E−03 | 6.65E−03 | 6.98E−03 | 6.96E−03 | 6.90E−03 |
| Lungs | 2.93E−03 | 2.93E−03 | 2.90E−03 | 2.93E−03 | 2.92E−03 | 2.92E−03 |
| Pancreas | 2.65E−02 | 2.64E−02 | 2.51E−02 | 2.66E−02 | 2.64E−02 | 2.62E−02 |
| Prostate | 7.79E−03 | 7.76E−03 | 7.36E−03 | 7.81E−03 | 7.78E−03 | 7.70E−03 |
| Salivary Glands | 7.20E−05 | 7.62E−05 | 1.06E−04 | 7.05E−05 | 7.15E−05 | 7.92E−05 |
| Red Marrow | 2.69E−03 | 2.69E−03 | 2.59E−03 | 2.69E−03 | 2.69E−03 | 2.67E−03 |
| Osteogenic Cells | 4.37E−03 | 4.37E−03 | 4.20E−03 | 4.38E−03 | 4.37E−03 | 4.34E−03 |
| Spleen | 8.01E−03 | 7.99E−03 | 7.62E−03 | 8.03E−03 | 8.00E−03 | 7.93E−03 |
| Testes | 5.56E−04 | 5.55E−04 | 5.26E−04 | 5.58E−04 | 5.56E−04 | 5.50E−04 |
| Thymus | 1.30E−03 | 1.31E−03 | 1.35E−03 | 1.30E−03 | 1.30E−03 | 1.31E−03 |
| Thyroid | 4.35E−04 | 4.76E−04 | 7.75E−04 | 4.20E−04 | 4.30E−04 | 5.07E−04 |
| Urinary Bladder | 4.75E−03 | 4.74E−03 | 4.49E−03 | 4.77E−03 | 4.75E−03 | 4.70E−03 |
| Wall | | | | | | |
| Total Body | 3.54E−03 | 3.53E−03 | 3.37E−03 | 3.55E−03 | 3.54E−03 | 3.51E−03 |
| Effective dose | 2.25E−02 | 2.25E−02 | 2.21E−02 | 2.25E−02 | 2.25E−02 | 2.24E−02 |
| (mSv/MBq) | | | | | | |

FIG. 1 shows the biodistribution in coronal and sagittal images of SPECT/CT scans of Patient 1 with GERD and four EoE patients (Patients 2-5) obtained approximately 1 hour after oral administration of Tc99m-heparin. The radioactivity is shown in red; notably, Patients 1 and 4 showed no binding to the esophagus. Patient 2 showed radioactivity in both proximal and distal esophagus (not mid esophagus). Patient 3, with severe EoE, showed marked radioactivity binding throughout the entire esophagus. Patient 5 showed bound radioactivity in the upper esophagus. Thus, radioactivity deposition is apparent in three patients with EoE and was prominent in Patient 3 on both coronal and sagittal images. The findings shown in FIGS. 10 and 11 were obtained at approximately one hour after swallowing and about 8:00 in the morning; binding of Tc99m-heparin to the esophagus was not detectable once patients had eaten food, at about 10:00, midmorning.

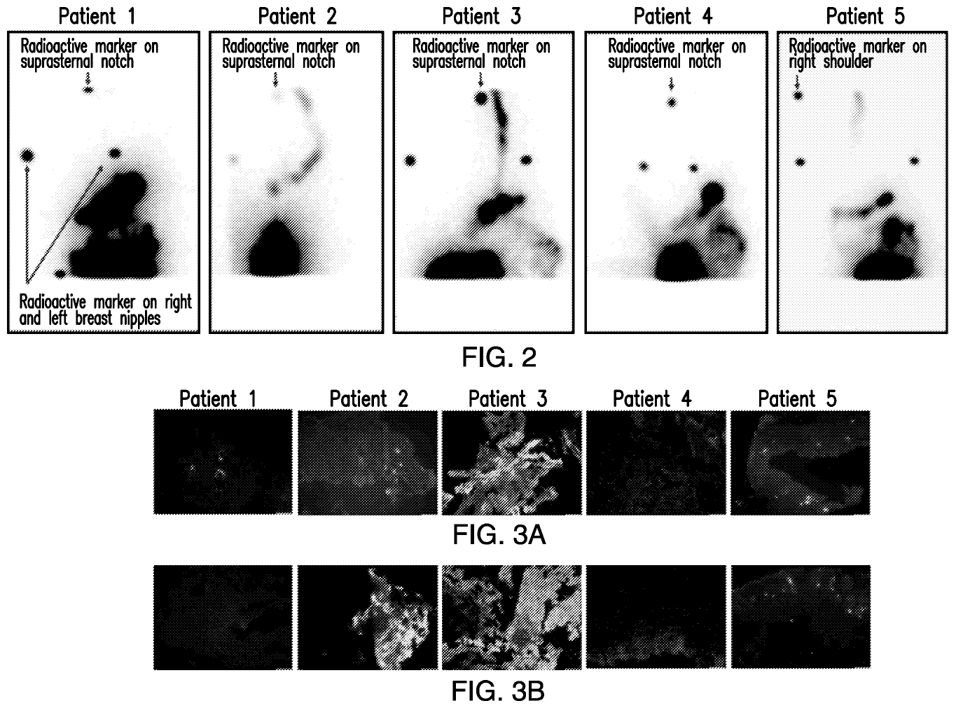

FIG. 2 shows the esophageal biodistribution in the individual patients at approximately 1 hour by SPECT analysis, without the CT component which was included in FIG. 1, using OsiriX DICOM Viewer software. As in FIG. 1, images were obtained after patients had swallowed Tc99m-heparin over a 15-minute time period and then swallowed 100 ml water (as a wash to remove weakly bound Tc99m-heparin). Images are labeled to identify markers including suprasternal notch (on Patients 1, 2, 3 and 4), right shoulder (on Patient 5) and breast/nipples (evident on the images for Patients 1, 3, 4, and 5 while for Patient 2, the left breast/nipple marker is obscured). Esophageal binding of Tc99m-heparin is clearly observed in Patients 2, 3 and 5, with localization also observed in the stomach, at the end of the esophagus below the breast/nipple markers, and in the intestines. These SPECT images show more distinctly the differences among the patients; Patient 1 and 4 show no evidence of radioactivity binding in the esophagus whereas Patients 2, 3 and 5 do show binding.

Table 3 summarizes findings from the patients including visual ratings of Tc99m-heparin binding in proximal, mid and distal esophageal segments, EREFS scores from endoscopy totaled for each segment, peak eosinophil counts per HPF on histopathological analyses of biopsies from each segment, measurements of bound radioactivity and eMBP-1 immunostaining scores on biopsy specimens from proximal and distal esophageal segments (biopsy specimens from mid esophagus were not obtained for eMBP-1 immunostaining). FIG. 3A-B shows representative fluorescent photomicrographic images of the eMBP-1 immunostaining in the proximal (a) and distal (b) esophageal biopsies from the individual patients.

With respect to the visual Tc99m-heparin binding scores, there was agreement in the intensity gradings among the three evaluators, who each performed evaluations independently without knowledge of others' grading. Patient 1, with GERD, had no evidence of eosinophil infiltration and no deposition of eMBP-1 (Table 3 and FIG. 3). Patient 2 had EoE, particularly involving the distal esophagus demonstrating peak eosinophil count of 42/HPF. Although the eosinophil counts were not increased proximally, immunostaining showed evidence of eMBP-1 deposition in concert with relatively prominent endoscopic findings of EoE, indicating the presence of disease activity proximally (Table 3 and FIG. 3). Patient 3 suffered from severe EoE, with peak eosinophils counts greater than 75/HPF (note that a peak eosinophil count greater than 15/HPF is indicative of EoE) in the three regions, as well as maximal grade 3+ eMBP-1 immunostaining (Table 3 and FIG. 3). Patient 4 had been diagnosed with EoE and was on treatment at the time of the imaging; the findings across the various parameters that were tracked indicated that he was free of inflammation at the time of the study (EREFS scores of 0, no increased peak eosinophil counts, and no evidence of eMBP-1 staining in biopsy specimens, Table 3 and FIG. 3). Patient 5 had EoE in the proximal and mid esophagus (peak eosinophil count of 91/HPF in proximal and 32/HPF in mid esophageal biopsy specimens) and 0.5+ eMBP-1 immunostaining (Table 3 and FIG. 3).

The geometric mean radioactivity counts from anterior and posterior whole-body images also are presented in Table 3. Patients 1 and 4, with no eosinophils by histopathology, had radioactivity counts of 6625 and 5010, respectively, whereas Patient 3, who had high eosinophil counts in the three segments, showed the greatest radioactivity counts in the esophagus with a geometric reading of 66,323 counts; over 10 times greater than Patients 1 and 4.

TABLE 3
Summary of esophageal findings in five individual patients, including disease
state, visual scores of Tc99m-heparin binding, endoscopy scores, radioactivity
counts from planar images and eMBP-1 immunostaining scores.

| | | Visual | | Peak | Geometric | |
|---|---|---|---|---|---|---|
| | | Tc99m-heparin | EREFS Summed | Eosinophil | Counts of | eMBP-1 |
| Patient | Disease | binding | Endoscopy Scores | Counts | Planar Esophagus | Scores |
| Number | State | (P, M, D)* | (P, M, D)# | (P, M, D) | Images** | (P, D)## |
| 1 | Non-EoE | (0, 0, 0) | (0, 0, 0) | (0, 0, 0) | 6,626 | (0, 0) |
| | (GERD) | | | | | |
| 2 | EoE | (2, 1, 3) | (4, 4, 5) | (0, 5, 42) | 18,363 | (0.5+, 2-3+) |
| 3 | EoE | (4, 4, 2) | (4, 4, 4) | (80, 125, 143) | 66,323 | (3+, 3+) |
| 4 | EoE | (0, 0, 0) | (0, 0, 0) | (0, 0, 0) | 5,010 | (0, 0) |
| 5 | EoE | (2, 1, 0) | (4, 3, 2) | (91, 32, 0) | 16,010 | (0.5+, 0.5+) |

*P = Proximal, M = Mid and D = Distal esophageal segments.
#Sums of EREFS scores from endoscopy: E: edema, R: rings, E: exudates, F: furrows, S: strictures. For example, Patient 3 had scores of (E1R0E2F1S0, E1R0E2F1S0, E1R0E2F1S0 for the proximal, mid and distal esophageal segments), and these scores are totaled for each segment to derive the recorded numbers (4, 4, 4).
**Geometric mean counts from anterior and posterior planar images 2 hours after oral administration.
##eMBP-1 score by immunofluorescence localization

Correlation analyses testing the associations between Tc99m-heparin binding and markers of inflammation showed significant relationships: (1) Between peak eosinophils/HPF and visual grading scores of Tc99m-heparin binding intensity, rs=+0.84 p=0.001; (2) Between visual grading scores of Tc99m-heparin binding intensity and eMBP-1 immunostaining grades, rs=+0.87 p=0.001; (3) Between geometric mean radioactivity counts of planar images in approximately similar regions of interest and eMBP-1 immunostaining grades, rs=+0.98 p=0.005; and (4) Between EREFS summed endoscopy scores and visual grading scores of Tc99m-heparin binding grading, rs=+0.91 p<0.001.

The experiments described herein were carried out to study the distribution and dosimetry of Tc99m-heparin in patients with EoE and a patient without EoE. The results indicate that swallowed Tc99m-heparin transiently binds to the esophagus of patients with active EoE who are experiencing eosinophil-related inflammation, as shown by eosinophil infiltration and by deposition of eMBP-1 in tissue biopsy specimens. In contrast, no such binding was detected in two patients who did not have evidence of eosinophil-related esophageal inflammation. In the patients tested, the swallowed Tc99m-heparin passed through the gastrointestinal tract and little was absorbed. Therefore, dosimetry was determined, in large part, by the rate of passage through the gastrointestinal tract, and the right and left colon received the greatest radioactive exposure. The radiation dosimetry is comparable to other orally administered Tc99m agents used daily in nuclear medicine departments to evaluate gastric emptying.

The results shown in Table 3 and FIGS. 1-3 indicate that the biodistribution of Tc99m-heparin binding correlates with markers of eosinophil-related inflammation in patients with EoE. The esophageal regions showing the most prominent Tc99m-heparin binding also showed eosinophil infiltration and diffuse tissue deposition of eMBP-1 in the biopsies. Further, there was a significant association between the occurrence of eosinophil-related inflammation assessed by histopathology and eMBP-1 immunostaining in the tissues and binding of Tc99m-heparin.

Comparison of FIGS. 1 and 2 indicates that the SPECT images, along with the anatomical markers, are sufficient for orientation, identification and inspection of the esophagus; therefore, performance of CT may not be necessary or can be performed with minimal radiation exposure to identify esophageal Tc99m-heparin binding and, importantly, reduce overall radiation exposure. Tc99m-heparin has been employed in earlier studies to identify myocardial damage (Kulkarni P V, et al. J Nucl Med 1978; 19:810-5; and Duska F, et al. Nuklearmedizin 1985; 24:111-4) and to localize blood clots; (Utne H E, et al. Application of ^{99m}Tc-labelled heparin. Eur J Nucl Med 1981; 6:237-40; and Kitschke B, et al. Int J Nucl Med Biol 1984; 11:235-41) however, this is the first use of swallowed Tc99m-heparin to detect eosinophil-related inflammation in EoE.

This study shows the overall biodistribution of Tc99m-heparin, its passage through the gastrointestinal tract, and its binding to eMBP-1 in areas of esophageal inflammation. Furthermore, the Tc99m-heparin employed in this study, 88 mg heparin and 30 mCi Technetium99m, resulted in an excess of unlabeled “cold” heparin; calculations of the mole ratios of Tc99m-labeled and cold heparin indicate that 1/100,000 heparin molecules was radiolabeled. Thus, reducing the proportion of heparin in the formulation results in more favorable binding of Tc99m-labeled heparin to eMBP-1 in tissues, as confirmed by in vitro studies.

As described herein, heparin used in the patient studies was labelled with 30mCi Technetium^{99m} (to maximize clear signal) and 88 mg heparin (as prior studies had suggested that this was well tolerated by patients) (Saffari H, et al. J Allergy Clin Immunol 2014; 133:1728-34 el). However, this quantity of heparin likely is in excess of what is needed. For example, calculation of the mole ratio of Technetium^{99m}-labeled heparin and unlabeled heparin reveals that approximately 1 heparin molecule in every 100,000 molecules carries a radioactive label. In other words, for every radiolabeled (“hot”) heparin molecule (that can give a positive response by binding to eMBP-1), there are 99,999 unlabeled (“cold”) heparin molecules that compete for the binding site on eMBP-1 (and reduce binding to eMBP-1). Therefore, employing a ^{99m}Tc-heparin produced by using less unlabeled heparin should reduce “cold” inhibition of the binding to eMBP-1. In other words, using 1 mg of HMWH would reduce the “cold” inhibition by unlabeled heparin and improvise the signal. Table 4 shows the results of comparing 88 mg heparin labeled with 30 mCi Technetium^{m99} to 8 mg heparin labeled with 30 mCi Technetium^{m99} for binding to eMBP-1. The findings show that ^{99m}Tc-heparin produced using 88 mg heparin and 30mCi Technetium^{99m} was inferior to ^{99m}Tc-heparin produced using 8 mg heparin and 30mCi Technetium^{99m} in binding to eMBP-1 on the plate. For these experiments, wells were coated with 0.27 mg/mL eMBP-1 in phosphate saline buffer overnight; uncoated wells were used for comparison (background). The quantities of 99mTc-heparin labeled, 88 mg and 8 mg, are listed on the top and mCi Technetiumm99, 30 mCi, used for the labeling is listed at the left. Wells are washed and counted, and the results are listed as μCi bound. Ratio indicates average counts bound divided by background (BKG) counts bound to an uncoated well.

TABLE 4
Binding of heparin-Tcm99 to 96 well plates with eMBP-1 (MBP).

| | | 88 mg | | | 8 mg | | |
|---|---|---|---|---|---|---|---|
| | | Activity | | | Activity | | |
| Tc/Heparin | | (μCi) | Average | Ratio | (μCi) | Average | Ratio |
| 30 mCi | MBP | 6.56 | 5.44 | 1.33 | 17.96 | 15.84 | 11.56 |
| | MBP | 7.35 | | | 14.39 | | |
| | MBP | 2.41 | | | 15.16 | | |
| | BKG | 3.8 | 4.1 | | 1.45 | 1.37 | |
| | BKG | 3.4 | | | 1.19 | | |
| | BKG | 5.1 | | | 1.47 | | |

Currently, identification of eosinophil-related inflammation in the esophagus requires multiple esophageal biopsies followed by histopathological tissue examination with eosinophil enumeration. Prior studies showed that heparin labeled with ^{99m}Technetium (Tc99m) bound to tissue specimens from diseased esophagi of patients with eosinophilic esophagitis (EoE). Therefore, it was tested whether Tc99m-heparin could serve as an imaging probe to detect eosinophil-related inflammation in EoE patients.

The biodistribution and radiation dosimetry of Tc99m-heparin, oral administration in patients with and without EoE utilizing new image-based dosimetry models with explicit modeling of the esophagus was tested.

Freshly prepared Tc99m-heparin was administered orally to five patients. Radioactivity was measured in the esophagus and other abdominal organs by whole-body scintigraphy during the 24 hours post-administration. Following imaging procedures, endoscopic examinations and analyses of biopsy specimens were performed. The biodistribution of esophageal radioactivity was compared to eosinophil counts obtained by histopathological examination of biopsy tissues and to immunostaining for eosinophil granule major basic protein-1 (eMBP-1). Radioactivity values as percent of injected dose per organ were fit using Simulation, Analysis, and Modeling Software II (SAAM II), and time integrals of radioactivity were entered into the Organ Level INternal Dose Assessment/EXponential Modeling (OLINDA/EXM) 2.0®dyna software using the adult male model.

Oral administration of Tc99m-heparin was well tolerated in all five patients. Ninety percent or more of the radioactivity did not bind to the esophagus and passed through the gastrointestinal tract. The entire esophagus could be visualized dynamically during oral administration, and static images were captured. Radioactivity that bound to the esophagus was higher in patients with active EoE, than in patients without active disease and was associated with markers of eosinophil-related inflammation, including numbers of eosinophils per high power field (HPF) and cellular and extracellular localization of eMBP-1 by immunostaining.

As shown in this Example, oral administration of Tc99m-heparin was well tolerated, and the biodistribution of orally administered Tc99m-heparin is almost exclusively localized to the gastrointestinal tract. Radiation exposure was highest in the lower gastrointestinal tract and comparable to other orally administered diagnostic radiopharmaceuticals. This shows that Tc99m-heparin scintigraphy can be useful to assess eosinophil-related inflammation in the esophagus.

### Example 2 Heparin SEC Fractions Binding to EMBP-1 Via Surface Plasmon Resonance (SPR)

Different forms of heparin were tested to determine their binding to eMBP-1. Fractionated heparin was assessed by size exclusion chromatography.

Surface Plasmon Resonance. SPR analysis was conducted on the Bruker Scientific (Billerica, MA) MASS-1 instrument in HBS (HEPES buffered saline) running buffer (10 mM HEPES pH 7.4, 150 mM NaCl) using the low-charge density polycarboxylate hydrogel surface (HLC200M) from Xantec Bioanalytics (Duesseldorf, Germany). Approximately 1500-3000 response units (RUs) of eMBP-1 were immobilized on the surface via thiol coupling. Specifically, the carboxylate surface was first activated with 75 mM sulfo-NHS and 100 mM 1-Ethyl-3-[3-dimethylaminopropyl]carbodiimide hydrochloride (EDC) (4 min at 10 μL/min) followed by 50 mM 2-(2-pyridinyldithio)ethanamine (PDEA) in 30 mM sodium borate pH 8.0 (6 min at 10 μL/min). eMBP-1 (0.6 μM in 10 mM sodium acetate pH 5.25) was then immobilized (6 min at 10 μL/min) followed by capping with 50 mM cysteine in 1 M NaCl and 0.1 M NaAc, pH 4.0 (4 min at 10 μL/min). The steps were performed on both the ‘A’ and ‘B’ spots of each channel, except for the eMBP-1 immobilization, which was performed on the ‘B’ spots, leaving the ‘A’ spots as in-line controls. Binding of the eMBP-1 ligands (fractionated and unfractionated heparin and enoxaparin) was tested in triplicate at equal mass/volume ratios (ranging from −0.2 μg/mL to 12 μg/mL) at 25 μL/min with 8 min association and 30 min dissociation phases. In between ligand injections, regeneration of the surface was performed with two 5 s pulses of 6 M guanidine-HCl (50 μL/min). Data were corrected by subtracting the in-line blank surface (B−A) and double-referenced using running buffer injections which were performed before each ligand injection.

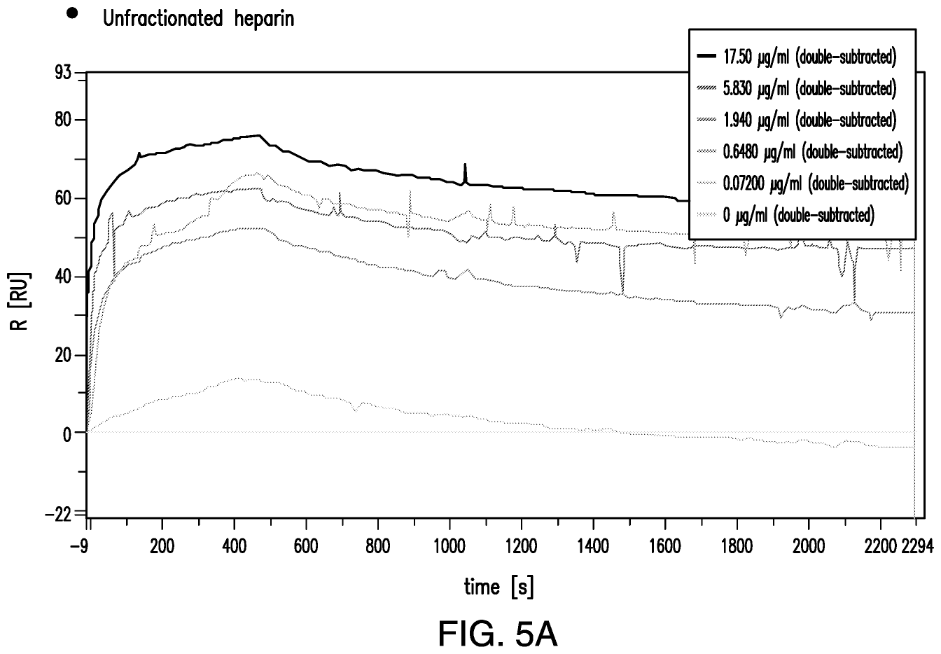

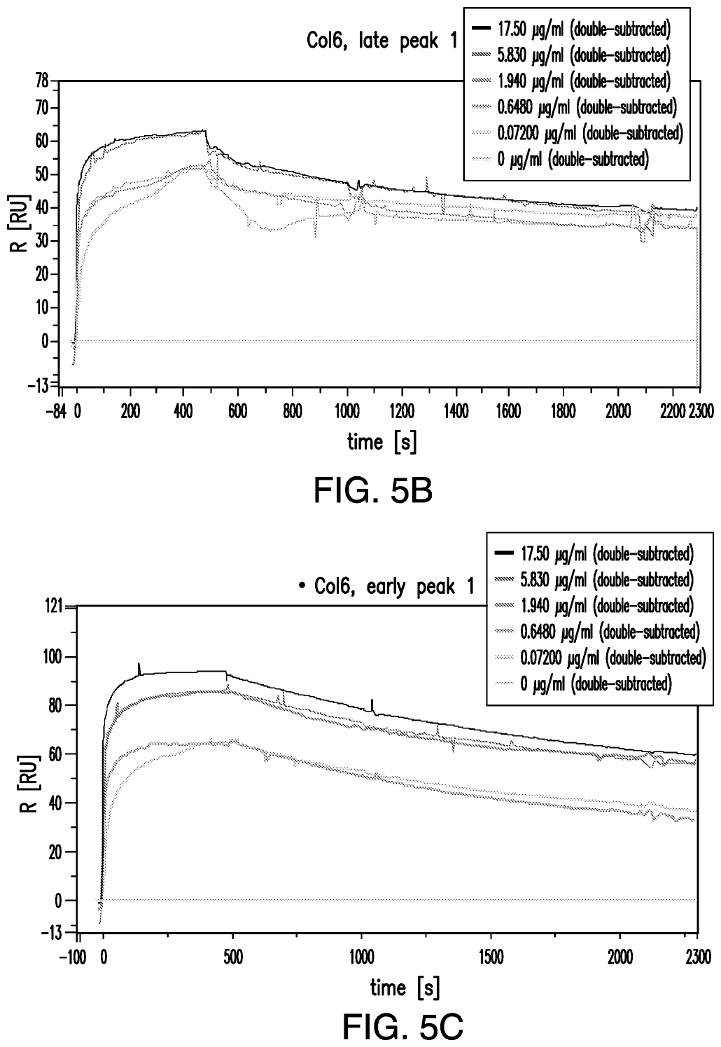

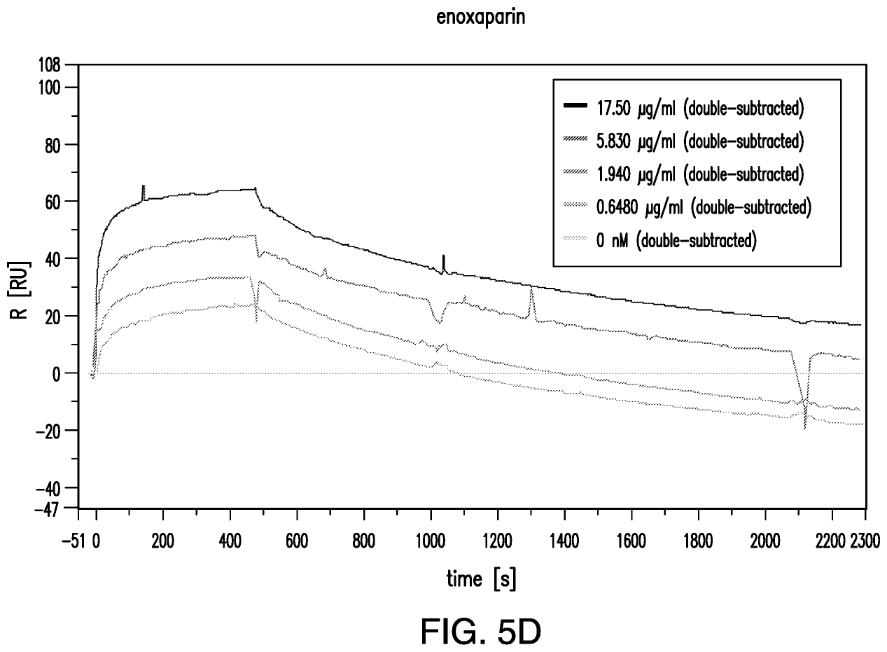

FIGS. 4-5 shows the analyses of heparin binding to immobilized MBP by surface plasmon resonance. More specifically, FIG. 4 shows that most intense binding is by heparins eluting from the BioGel P60 column in peak 1. In contrast, heparin in peak 2 bound poorly to MBP. FIG. 5A shows varying concentrations of unfractionated heparin using pharmaceutical grade heparin (commonly employed for patient treatment). FIGS. 5B-D show binding of differing concentrations of fractions from the BioGel P60 column. FIG. 6 show the fractionation of heparin by gel permeation chromatography.

As part of these studies, a variety of molecules including low molecular weight heparin (approximately 5 kDa), were probed. However, unfractionated heparin (e.g. unfractionated USP pharmaceutical grade heparin) bound better to eMBP-1 then did low molecular weight heparin. Additional studies were conducted on different forms of heparin. These studies generated a series of heparin molecules, and analyses by surface plasmon resonance showing that high molecular weight heparin (estimated 20 kDa) binded more avidly to eMBP-1 than various lower molecular weight heparins.

These findings evidence the utility of high molecular weight heparin labelled with technetium-99M for localization of eosinophilic inflammation in eosinophil-related diseases. Moreover, with the knowledge that heparin can neutralize the toxic effects of eMBP-1 and the known association of eMBP-1 tissue deposition with organ dysfunction, these findings evidence that the high molecular weight forms of heparin may be a preferred agent for eMBP-1 neutralization, and, thus a therapeutic agent for eosinophilic diseases. High molecular weight forms, based on their striking affinity and assumed ability to neutralize eMBP-1, can be preferred reagents for localization of eosinophilic inflammation and for neutralization of the toxic effects of MBP and thus treatment of eosinophil-related diseases.

Presently unfractionated USP heparin utilized for patient care is labeled with technetium-99M for localization of eosinophilic inflammation. The data described herein show the utilization of this technology to identify inflammation in eosinophilic esophagitis. Initial studies tested 88 mg of USP heparin labelled with 30 mCi of technetium-99M. More recent studies employed 2.8 mg of heparin and 3 mCi of technetium-99M. The observation that high molecular weight heparin has such marked avidity for eMBP-1 suggests that as little as 1 mg of high molecular weight heparin, or less, can be used to label with technetium99M. To support this notion, it was assumed that a greater percentage of the high molecular weight heparin will bind to eMBP-1 in tissues as compared to the case of low molecular weight heparin or unfractionated heparin and thus a greater percentage of radioactivity will be localized to these sites. Greater binding of technetium-99M bound heparin should permit use of less radioactivity overall with the assumption that a higher proportion will bind to areas of inflammation.

### Example 3 Purification of High Molecular Weight Heparin by Gel Filtration

Preservative-free, medicinal grade heparin was fractionated on a column of BioGel P-60 (Bio-Rad Laboratories) 1.2 cm by 95 cm in 0.5 M NH₄HCO₃ at 20° C. Results are shown in FIG. 11. The column was calibrated with blue dextran (molecular mass ˜2,000 kDa) and vitamin B12 (molecular mass 1356 Da); blue dextran eluted at 33 ml (the void volume of the column) and vitamin B12 eluted at 100 ml. The results shown in FIG. 11 were repeated with excellent agreement.

Chromatography of medicinal grade heparin on BioGel P-60. Medicinal grade heparin, USP, 60 ml, 2000 units per ml, was lyophilized (weight of powder 0.9 gm), and the powder was taken up in water, and a total of 2.4 ml was applied to the column. Heparin was detected by absorbance at 232 nm. Blue dextran eluted at 33 ml and vitamin B12 eluted at 100 ml. Heparin was contained in fractions eluting between 33 ml and 80 ml. The absorbance after ˜100 ml was due to low molecular weight components that were not characterized. High molecular weight heparins were contained in the fractions eluting from the void volume, 33 ml, to approximately 50 ml.

It will be apparent to those skilled in the art that various modifications and variations can be made in the present invention without departing from the scope or spirit of the invention.

Other aspects of the invention will be apparent to those skilled in the art from consideration of the specification and practice of the invention disclosed herein. It is intended that the specification and examples be considered as exemplary only, with a true scope and spirit of the invention.

What is claimed:

1. A composition comprising: an effective amount of heparin having an average molecular weight from about 20 kDa to about 40 kDa, wherein at least 50% of heparin chains in the heparin have a molecular weight of at least 20 kDa; and a pharmaceutically acceptable excipient.

2. The composition of claim 1, wherein the heparin comprises an average molecular weight of at least 20 kDa.

3. The composition of claim 2, wherein the heparin comprises an average molecular weight of at least 30 kDa.

4. The composition of claim 3, wherein the heparin comprises an average molecular weight of at least 40 kDa.

5. The composition of claim 1, wherein at least 60% of heparin chains in the heparin have a molecular weight of at least 20 kDa.

6. The composition of claim 5, wherein at least 70% of heparin chains in the heparin have a molecular weight of at least 20 kDa.

7. The composition of claim 1, wherein the effective amount of heparin is about 3 mg.

8. The composition of claim 7, wherein the effective amount of heparin is about 1 mg.

9. The composition of claim 8, wherein the effective amount of heparin is about 0.5 mg.

10. The composition of claim 1, wherein the heparin is configured to bind to one or more eosinophil granule proteins.

11. The composition of claim 10, wherein the binding affinity of the heparin for the one or more eosinophil granule proteins is greater than the binding affinity of a low molecular weight heparin for the one or more eosinophil granule proteins.

12. The composition of claim 10, wherein the one or more eosinophil granule proteins comprise one or more of major basic protein 1 (MBP-1), major basic protein 2 (MBP-2), eosinophil derived neurotoxin (EDN), eosinophil cationic protein (ECP), and eosinophil peroxidase (EPO).

13. The composition of claim 1, wherein the composition is configured to be administered orally.

14. The composition of claim 1, wherein the composition is therapeutically effective to treat eosinophilic esophagitis.

15. The composition of claim 1, further comprising a therapeutic agent conjugated to the heparin.

16. The composition of claim 15, wherein the therapeutic agent is a glucocorticoid.

17. The composition of claim 1, further comprising a radiolabeled contrast agent conjugated to the heparin.

18. The composition of claim 18, wherein the radiolabeled contrast agent is ^{99m}Tc.

19. A method of treating a tissue exhibiting eosinophil-related inflammation in a subject, the method comprising: administering to the subject a composition comprising a therapeutically effective dose of heparin having an average molecular weight from about 20 kDa to about 40 kDa, wherein at least 50% of heparin chains in the heparin have a molecular weight of at least 20 kDa, and a pharmaceutically acceptable excipient, wherein the heparin binds to one or more eosinophil granule proteins in the tissue to reduce the eosinophil-related inflammation.

20. A method of reducing eosinophil-related inflammation in a tissue, the method comprising: administering to a subject a composition comprising a therapeutically effective dose of heparin having an average molecular weight from about 20 kDa to about 40 kDa, wherein at least 50% of heparin chains in the heparin have a molecular weight of at least 20 kDa, and a pharmaceutically acceptable excipient, wherein the heparin binds to one or more eosinophil granule proteins in the tissue to reduce the eosinophil-related inflammation.

21. The method of claim 19 or 20, wherein the heparin comprises an average molecular weight of at least 20 kDa.

22. The method of claim 21, wherein the heparin comprises an average molecular weight of at least 30 kDa.

23. The method of claim 22, wherein the heparin comprises an average molecular weight of at least 40 kDa.

24. The method of claim 19 or 20, wherein at least 60% of heparin chains in the heparin have a molecular weight of at least 20 kDa.

25. The method of claim 24, wherein at least 70% of heparin chains in the heparin have a molecular weight of at least 20 kDa.

26. The method of claim 19 or 20, wherein the therapeutically effective dose of heparin is about 3 mg.

27. The method of claim 26, wherein the therapeutically effective dose of heparin is about 1 mg.

28. The method of claim 27, wherein the therapeutically effective dose of heparin is about 0.5 mg.

29. The method of claim 19 or 20, wherein the binding affinity of the heparin for the one or more eosinophil granule proteins is greater than the binding affinity of a low molecular weight heparin for the one or more eosinophil granule proteins.

30. The method of claim 19 or 20, wherein the one or more eosinophil granule proteins comprise one or more of major basic protein 1 (MBP-1), major basic protein 2 (MBP-2), eosinophil derived neurotoxin (EDN), eosinophil cationic protein (ECP), and eosinophil peroxidase (EPO).

31. The method of claim 19 or 20, wherein the composition is configured to be administered orally.

32. The method of claim 19 or 20, wherein the composition is therapeutically effective to treat eosinophilic esophagitis.

33. The method of claim 19 or 20, further comprising a therapeutic agent conjugated to the heparin.

34. The method of claim 33, wherein the therapeutic agent is a glucocorticoid.

35. The method of claim 19 or 20, further comprising a radiolabeled contrast agent conjugated to the heparin.

36. The method of claim 35, wherein the radiolabeled contrast agent is ^{99m}Tc.

37. A method of producing a medical image of an organ in a subject, the method comprising:
detecting an eosinophil granule protein in the mucosal tissue of the organ in a subject, comprising administering to a subject radiolabeled heparin under conditions wherein the radiolabeled heparin binds to an eosinophil granule protein to form a radiolabeled heparin/eosinophil granule protein complex, and
detecting the radiolabeled heparin/eosinophil granule protein complex in the mucosal tissue of the organ, whereby detecting the radiolabeled heparin/eosinophil granule protein complex in the mucosal tissue of the organ produces a medical image of the organ in the subject.

38. A method of diagnosing eosinophilic esophagitis in a subject, the method comprising:
detecting an eosinophil granule protein in the mucosal tissue of the esophagus in a subject, comprising administering to a subject radiolabeled heparin under conditions wherein the radiolabeled heparin binds to an eosinophil granule protein to form a radiolabeled heparin/eosinophil granule protein complex, and
detecting the radiolabeled heparin/eosinophil granule protein complex in the mucosal tissue of the esophagus, whereby detecting the radiolabeled heparin/eosinophil granule protein complex in the mucosal tissue of the esophagus diagnoses eosinophilic esophagitis in the subject.

39. A method of detecting a change in eosinophilic esophagitis in a subject diagnosed with eosinophilic esophagitis, the method comprising:
a) producing a first medical image of the esophagus in a subject diagnosed with eosinophilic esophagitis according to the method of claim 37;
b) producing a second medical image of the esophagus in the subject of step (a), according to the method of claim 37; and
c) comparing the medical image of step (b) with the medical image of step (a), whereby detecting a change in the medical image of step (b) compared to the medical image of step (a) detects a change in eosinophilic esophagitis in the subject.

40. A method of detecting eosinophil degranulation in a subject, the method comprising:
detecting an eosinophil granule protein in a subject, comprising administering to a subject radiolabeled heparin under conditions wherein the radiolabeled heparin binds to an eosinophil granule protein to form a radiolabeled heparin/eosinophil granule protein complex, and
detecting the radiolabeled heparin/eosinophil granule protein complex, whereby detecting the radiolabeled heparin/eosinophil granule protein complex detects eosinophil degranulation in the subject.

41. A method of delivering a therapeutic agent to a diseased organ, the method comprising, administering a therapeutically effective amount of a composition comprising heparin conjugated to a therapeutic agent to a subject.

42. The method of any one of claims 37-41, wherein the heparin has an an average molecular weight from about 20 kDa to about 40 kDa, and wherein at least 50% of heparin chains in the heparin have a molecular weight of at least 20 kDa.

43. A method of treating eosinophilic-related inflammation in a subject, the method comprising, administering a therapeutically effective amount of a composition comprising an effective amount of heparin having an average molecular weight from about 20 kDa to about 40 kDa, wherein at least 50% of heparin chains in the heparin have a molecular weight of at least 20 kDa; and a pharmaceutically acceptable excipient to the subject.

44. The method of claim 42 or 43, wherein the heparin comprises an average molecular weight of at least 20 kDa.

45. The method of claim 44, wherein the heparin comprises an average molecular weight of at least 30 kDa.

46. The method of claim 45, wherein the heparin comprises an average molecular weight of at least 40 kDa.

47. The method of claim 42 or 43, wherein at least 60% of heparin chains in the heparin have a molecular weight of at least 20 kDa.

48. The method of claim 47, wherein at least 70% of heparin chains in the heparin have a molecular weight of at least 20 kDa.

49. The method of claim 41 or 43, wherein the therapeutically effective dose of heparin is about 3 mg.

50. The method of claim 49, wherein the therapeutically effective dose of heparin is about 1 mg.

51. The method of claim 50, wherein the therapeutically effective dose of heparin is about 0.5 mg.

52. The method of claim 42 or 43, wherein the heparin is configured to bind to one or more eosinophil granule proteins.

53. The method of claim 52, wherein the binding affinity of the heparin for the one or more eosinophil granule proteins is greater than the binding affinity of a low molecular weight heparin for the one or more eosinophil granule proteins.

54. A method of treating eosinophilic-related inflammation in a subject, the method comprising, administering a therapeutically effective amount of a composition comprising heparin conjugated to a therapeutic agent to a subject.

55. The method of claim 37 or 41, wherein the organ is an ovary, a breast, a brain, a muscle, a heart, a lung, a stomach, a proximal large intestine, a distal large intestine, a small intestine, a pancreas, a thyroid, skin, an eye, a testicle, a thymus, a gallbladder, a uterus, an esophagus or a major blood organ.

56. The method of claim 55, wherein the major blood organ is the liver, spleen, kidneys, or bladder.

57. The method of any of claims 37-40 and 52-53, wherein the eosinophil granule protein is major basic protein 1 (MBP-1), major basic protein 2 (MBP-2), eosinophil derived neurotoxin (EDN), eosinophil cationic protein (ECP), or eosinophil peroxidase (EPO).

58. The method of claim 57, wherein the eosinophil granule protein is MBP-1.

59. The method of any of claim 37-40 or 55-57, wherein the radiolabel of the radiolabeled heparin is ^{99m}Tc.

60. The method of any of claim 37-40 or 55-58, wherein the radiolabeled heparin is administered to the subject orally.

61. The method of claim 60, wherein the subject swallows the radiolabeled heparin through one or more swallows.

62. The method of claim 61, wherein the radiolabeled heparin is administered orally to the subject in 1 ml aliquots over 15 minutes.

63. The method of any of claim 37-40 or 55-62, further comprising a washing step.

64. The method of claim 63, wherein the washing step comprises the subject swallowing a liquid after the one or more swallows of the radiolabeled heparin.

65. The method of claim 64, wherein the liquid is water.

66. The method of claim 64, wherein the washing step is performed before, during or after the production of a medical image.

67. The method of claim 62, wherein the administration of the liquid comprises the subject swallowing the liquid through one or more swallows.

68. The method of any of claims 64-67, wherein the liquid is administered in a volume of 1 to 100 ml.

69. The method of any of the preceding claims, wherein the heparin or heparin portion of the radiolabeled heparin is high molecular weight heparin, low molecular weight heparin or unfractionated heparin.

70. The method of claim 69, wherein the heparin or heparin portion of the radiolabeled heparin is high molecular weight heparin.

71. The method of claim 70, wherein the high molecular weight heparin is administered in an amount less than 1 mg.

72. The method of claim 70, wherein the high molecular weight heparin is administered in an amount ranging from 0.1 mg to 1 mg.

73. The method of any of the preceding claims, wherein the radiolabel of the radiolabeled heparin is administered in an amount ranging from 0.3 mCi to 3 mCi.

74. The method of any of the preceding claims, wherein the subject is a human.

75. The method of any of claim 37-40 or 55-74, wherein the radiolabeled heparin/eosinophil granule protein complex is detected using one or more of single-photon emission computed tomography (SPECT), positron emission tomography (PET) scans, X-ray, conventional or computed tomography (CT), a combination of SPECT and CT, or magnetic resonance imaging (MRI).

76. The method of any of claim 37-40 or 55-74, wherein the medical image is three-dimensional.

77. The method of any of claim 37-40 or 55-74, wherein the medical image is two-dimensional.

78. The method of any of claims 41-56, wherein the therapeutic agent is a glucocorticoid.

79. The method of claim 78, wherein the glucocorticoid is mometasone, fluticasone, budesonide, prednisone or solumedrol.

80. The method of any of claim 41-56 or 78-79, further comprising identifying the subject in need thereof.

81. The method of claim 80, wherein the subject in need thereof is identified by any one of the methods of claims 37-40, 43 and 54.

82. The method of any of claim 41-56 or 78-81, wherein the composition is administered to the subject orally, intravenously, optically or topically.
